(19) United States
(12) Patent Application Publication
(10) Pub. No.: US 2022/0079187 A1
(43) Pub. Date: Mar. 17, 2022

(54) NON-DAIRY ANALOGS AND BEVERAGES WITH DEAMIDATED PLANT PROTEINS AND PROCESSES FOR MAKING SUCH PRODUCTS

(71) Applicant: RIPPLE FOODS, PBC, Berkeley, CA (US)

(72) Inventors: Amanda STILES, Berkeley, CA (US); Celia HOMYAK, Berkeley, CA (US)

(73) Assignee: RIPPLE FOODS, PBC, Berkeley, CA (US)

(21) Appl. No.: 17/421,480

(22) PCT Filed: Jan. 17, 2020
(86) PCT No.: PCT/US20/14045

§ 371 Date: Jul. 8, 2021

Related U.S. Application Data
(60) Provisional application No. 62/794,043, filed on Jan. 18, 2019.

(51) Int. Cl. A23J 3/34; C12P 21/00; A23C 11/10; A23J 3/14; A23L 2/66; A23L 2/60; A23L 33/15; A23L 33/16; A23F 5/24; A23L 11/60; A23L 33/185

(52) U.S. Cl. CPC A23J 3/346; C12P 21/00; A23C 11/103; A23J 3/14; A23L 2/66; A23V 2002/00; A23L 33/15; A23L 33/16; A23F 5/243; A23L 11/60; A23L 33/185; A23L 2/60

(57) ABSTRACT

The present disclosure is related to non-dairy analogs and beverage formulations comprising a deamidated refined protein component and method of using such products. The non-dairy analogs and beverage formulations exhibit improved properties such as decreased precipitation of the refined protein component. In certain embodiments, these non-dairy analogs can exhibit improved stability when used as a substitute for a dairy product in food products including but not limited to yogurt, sour cream, ice cream, coffee creamer, heavy cream, whipping cream, pudding, soft cheese, or hard cheese. In certain embodiments, the non-dairy analogs exhibit substantially reduced or no visible feathering when added to hot beverages such as coffee or tea.

### CROSS-REFERENCE TO RELATED APPLICATIONS

This application claims priority benefit of U.S. Provisional Patent Application 62/794,043, filed Jan. 18, 2019, which is hereby incorporated by reference herein.

### FIELD

The present disclosure relates to food products, such as non-dairy analogs or beverage formulations, that are derived substantially from or wholly from non-animal sources, and wherein at least a portion of the protein used in the food product is a deamidated refined protein, such as pea protein, that exhibits improved stability, dispersibility, and/or solubility

### BACKGROUND

Consumers often add food products such as dairy creamers to beverages such as coffee or tea. Dairy creamers are often made with dairy milk and/or dairy cream. Dairy creamers are desired by many consumers because of the milky flavor and creamy texture they add to the beverage. In addition, these dairy creamers tend to mix well with the beverage. Dairy creamers, and other dairy-based products, however have many qualities that consumers find disadvantageous, including but not limited to, the presence of saturated fat, the amount of fat, the presence of cholesterol, and/or the presence of lactose. For example, many people would prefer a non-dairy alternative to these dairy creamers because of the high fat and calorie levels. In addition, many people are not able to tolerate such products due to lactose intolerance and/or prefer not to consumer animal-based products.

There are known non-dairy analogs, for example a soy protein. However, these alternative non-dairy analogs suffer from several drawbacks, for example, a thin and chalky mouth feel, a green or beany flavor, undesirable color and so forth. In particular, one set of problems with existing non-dairy analogs is the product's relative lack of stability, dispersibility, and/or solubility when added to a such as coffee. When adding dairy milk or dairy creamer to coffee it typically dissolves and disperses well, remains stable within solution, and provides a whitening or creamy look to the coffee. A problem with existing non-dairy analogs is that they exhibit feathering when added to a beverage such as coffee or tea. Feathering is typically described as the presence of particles due to coagulation or precipitation of proteins within the beverage. Feathering imparts an undesirable visual experience and/or an undesirable mouthfeel experience for the consumer of the beverage in which it occurs. Proteins are typically a substantial component of non-dairy analogs and/or non-dairy milk products and their lack of stability, lack of dispersibility, and/or lack of solubility are believed to play a role in the feathering that occurs in these certain beverages.

Also, there are a wide range of animal-based protein containing beverage formulations (e.g., protein drinks, post-work-out drinks, vitamin drinks, exercise drinks, electrolyte drinks, fruit juice drinks, and iced tea drinks) for which consumers would desire a non-animal-based protein alternative (e.g., plant-based protein). For such beverage formulations it is highly desirable to minimize any precipitation of the protein component because consumers generally prefer a substantially transparent beverage without gritty or chalky solids present.

To reduce feathering in non-dairy analogs it has been proposed to include inorganic salts in such products. For example, it has been proposed to include dipotassium phosphate, sodium aluminum phosphate, and polyphosphates. However, the use of such inorganic salts has a negative impact on the taste, health and/or nutritional aspects of such non-dairy analogs.

Therefore, there exists an unmet need for non-dairy analogs for use in food beverages such as coffee or tea, and protein containing beverage formulations, that are stable, soluble, and/or exhibit minimal (or no) protein precipitation or feathering

The present disclosure is directed to solving these and other problems disclosed herein. The present disclosure is also directed to overcome and/or ameliorate at least one of the disadvantages of the prior art as will become apparent from the discussion herein.

### SUMMARY

As well as the embodiments discussed in the summary, other embodiments are disclosed in the specification, drawings, and claims. The summary is not meant to cover each and every embodiment; combination or variations are contemplated with the present disclosure.

Protein glutaminase may be used to deamidate plant proteins. It is an advantageous technical effect of such deamidated plant proteins that non-dairy analogs or beverage formulations incorporating at least a portion of deamidated plant proteins exhibit a significantly decreased protein precipitation, coagulation, and feathering, such as when added to acidic beverages (e.g., coffee or tea). The present disclosure provides food products, such as non-dairy analogs, that incorporate such deamidated plant proteins, methods of making such products and methods of using such products. The present disclosure is also directed to pointing out one or more advantages to using the products and/or methods disclosed herein.

In some embodiments the present disclosure is directed to methods for increasing the stability of plant proteins (e.g. pea proteins) using protein glutaminase to deamidate the plant protein. The deamidated plant proteins made using the methods can be used in non-dairy analogs that are mixed with acidic beverages, such as hot coffee or tea, or used in beverage formulations that contain plant protein.

In some embodiments, the present disclosure is directed to non-dairy analogs wherein at least a portion of the protein used in the non-dairy analog is a deamidated refined protein, wherein the presence of the deamidated refined protein results in increased solubility, increased dispersibility, and/or increased stability of the non-dairy analog when used as a substitute in a dairy product (e.g., yogurt, sour cream, creamers, and cheeses). In some embodiments, the non-dairy analog wherein at least a portion of the protein used in the non-dairy analog is a deamidated refined protein, is a creamer and exhibits reduced feathering when added to beverages (for example acidic beverages, such as coffee or tea).

In some embodiments, the present disclosure is directed to the use of a deamidated refined protein as an ingredient in a range of other food products, for example, yogurts, sour cream, milk, creamers, creams, and cheeses. The deamidated refined protein may be used as a separate ingredient in other food products and is not limited to use only in non-dairy analogs.

In some embodiments, the present disclosure is directed to a non-dairy analog, the non-dairy analog comprising: (a) a refined protein component in which at least a portion of the refined protein component is a deamidated refined protein component; (b) at least one lipid in which the at least one lipid is from a non-animal natural source; (c) at least one emulsifier; (c) water; and (d) a pH of between 4.0 and 10; optionally, a pH between 6.5 and 10.

In some embodiments, the present disclosure is directed to a non-dairy analog, the non-dairy analog comprising: (a) at least 0.2% by weight of a refined protein component in which at least 10% by weight of the refined protein component is a deamidated refined protein component; (b) between 1% by weight and 10% by weight of at least one lipid in which the at least one lipid is from a non-animal natural source; (c) between 0.01% by weight and 5% by weight of at least one emulsifier; (d) water; and (e) a pH of between 4.0 and 10; optionally, a pH between 6.5 and 10.

In some embodiments, the present disclosure provides a beverage formulation, wherein the formulation comprises: (a) a refined protein component in which at least a portion of the refined protein component is a deamidated refined protein component; (b) water or carbonated water; and (c) a pH of between 6 and 9.

In some embodiments of the beverage formulation, the formulation further comprises: (a) sugar and/or a carbohydrate; (b) at least one vitamin or mineral; (c) at least one lipid from a non-animal natural source; and/or (d) at least one emulsifier, and/or a hydrocolloid or gum. In some embodiments, the beverage formulation is selected from: a protein drink, a vitamin drink, a fruit juice drink, or an iced tea drink.

In some embodiments the present disclosure is directed to a non-dairy analog or a beverage formulation wherein the refined protein component comprises is at least 0.2%, 0.5%, 1%, 2%, 3%, 5%, 8% or 10% by weight of the non-dairy analog or the beverage formulation; optionally, between 0.2% to 10%, 0.5% to 10%, 1% to 5%, 3% to 8%, 3% to 4%, 3% to 8% or 2% to 4% by weight of the non-dairy analog or the beverage formulation.

In some embodiments the present disclosure is directed to a non-dairy analog or a beverage formulation wherein the wherein the deamidated refined protein component is at least 10%, 20%, 30%, 40%, 50%, 60%, 70%, 80%, 90% or 100% by weight of the total weight of the refined protein component; optionally, wherein the deamidated refined protein component is between 10% to 100%, 20% to 90%, 30% to 80%, 40% to 70%, 40% to 60%, or 30% to 50% by weight of the total weight of the refined protein component.

In some embodiments the present disclosure is directed to a non-dairy analog or a beverage formulation wherein the deamidated refined protein component is deamidated by glutaminase treatment; optionally, wherein the glutaminase treatment is carried out using a protein-glutaminase; optionally, wherein the protein-glutaminase is isolated or derived from C. proteolyticum. In some embodiments, the glutaminase treatment comprises incubation with at least 0.01 wt % and up to 10 wt % glutaminase relative to refined protein; optionally, incubation with between about 0.1 wt % and 15 wt %, between about 0.1 wt % and 10 wt %, between about 0.1 wt % and 5.0 wt %, between about 0.1 wt % and 2.5 wt %, or between about 0.1 wt % and 1.0 wt % glutaminase relative to refined protein.

In some embodiments the present disclosure is directed to a non-dairy analog or a beverage formulation wherein the refined protein component is sourced from a plant; optionally, sourced from a legume. Certain embodiments are directed to a non-dairy analog or beverage formulation wherein the refined protein component is sourced from a pea plant or a pea protein.

In some embodiments of the beverage formulation, the beverage: (a) exhibits less than 5%, 3%, 1% or 0.5% by weight precipitation of the refined protein component; and/or (b) exhibits less than 5%, 3%, 1% or 0.5% by volume precipitation of the refined protein component.

In some embodiments the present disclosure is directed to a method for producing a non-dairy analog, wherein the method comprises one or more of the following steps, in or out of order: a) obtaining at least one lipid from a non-animal natural source; b) obtaining at least one refined protein component from a non-animal natural source in which at least a portion of the refined protein component is a deamidated refined protein component; c) blending the at least one lipid and the at least one refined protein component with water to generate a mixture; and d) emulsifying at least a portion of the mixture to provide a non-dairy analog; whereby the quantities and proportions of the at least one lipid, and the at least one refined protein components are selected so as to provide a desired stability, dispersibility, and/or solubility of the non-dairy analog.

In some embodiments, the present disclosure is directed to a method for preparing a non-dairy analog, the method comprising: (a) generating an aqueous mixture at pH 7.5-8.5 and 35-60° C. comprising: an 0.5-5.0 wt % unmodified refined protein component from a non-animal natural source, and 0.3-1.0 wt % buffering salts; (b) adding to the mixture 0.1-1.0 wt % of a glutaminase and mixing at 35-60° C. for at least 1 hour, whereby at least a portion of the unmodified refined protein component is deamidated; (c) after mixing of step (b) adding to the mixture 2-10 wt % oil, 0.01-0.05 wt % gums, 0-1 wt % emulsifier, 0-1 wt % flavors and 0-2 wt % sugar; and (d) heating the mixture to 90° C. and subjecting to homogenization.

In some embodiments, the present disclosure is directed to a method for producing a beverage formulation, wherein the method comprises one or more the following steps, in or out of order: (a) obtaining a refined protein component from a non-animal natural source, in which at least a portion of the refined protein component is a deamidated refined protein component; (b) blending the deamidated refined protein component with water or carbonated water, and optionally, (i) with sugar and/or a carbohydrate, (ii) with at least one vitamin or mineral, (iii) with at least one lipid from a non-animal natural source, and/or (iv) with at least one emulsifier and/or a hydrocolloid or gum; and (c) adjusting the blended mixture to a pH of between 6 and 9.

### BRIEF DESCRIPTION OF THE DRAWINGS

Embodiments of the present disclosure are described, by way of example only, with reference to the accompanying figures.

FIG. 1 illustrates an exemplary embodiment. Images taken between 0-1 min. (top) and 5 min. (bottom) after addition of non-dairy analog to coffee. Non-dairy analogs contained either unmodified refined pea protein (left) or deamidated refined pea protein (right).

FIG. 2 depicts a plot of wet solids due to feathering in coffee produced by non-dairy analogs made with varying amounts of incubation with protein glutaminase which results in deamidation of the refined pea protein component.

### DETAILED DESCRIPTION

For the descriptions herein and the appended claims, the singular forms “a”, and “an” include plural referents unless the context clearly indicates otherwise. Thus, for example, reference to “a protein” includes more than one protein. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

The term “about” as used herein refers to greater or lesser than the value or range of values stated by 1/10 of the stated values, but is not intended to limit the value or range of values to only this broader definition. For instance, a value of “about 30%” means a value of between 27% and 33%. Each value or range of values preceded by the term “about” is also intended to encompass the embodiment of the stated absolute value or range of values. Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding (i) either or (ii) both of those included limits are also included in the invention. For example, “1 to 50,” includes “2 to 25,” “5 to 20,” “25 to 50,” “1 to 10,” etc.

Generally, the nomenclature used herein, and the techniques and procedures described herein include those that are well understood and commonly employed by those of ordinary skill in the art. Unless defined otherwise, all technical and scientific terms used herein have the same meaning as is commonly understood by one of ordinary skill in the art to which this disclosure pertains. It is to be understood that the terminology used herein is for the purpose of describing particular embodiments only and is not intended to be limiting. For purposes of interpreting this disclosure, the following description of terms will apply and, where appropriate, a term used in the singular form will also include the plural form and vice versa.

### Definitions

The term “dairy milk” as used herein refers to a white fluid secreted by the mammary glands of female mammals. Dairy milk consists of an emulsion of fat in an aqueous solution comprising proteins (e.g., casein, whey proteins), sugars, inorganic salts, and other ingredients. Suitable mammals from which dairy milk can be obtained include but are not limited to cow, sheep, goat, buffalo, donkey, horse, camel, yak, water buffalo, human, and other mammals. Dairy milk obtained from cow typically contains around 3.5% fat (whole cow milk). Fat levels can be reduced to standardized levels to obtain different grades of cow milk that comprise from 0% to 75% by weight of the fat present in whole cow milk, including but not limited to 2% cow milk (cow milk comprising 2% by weight of fat), 1% cow milk (cow milk comprising 1% by weight of fat), and skim cow milk (cow milk comprising no fat).

The term “non-dairy analog” as used herein refer to food products that can be used as a substitute for a dairy product but that is made from a non-dairy natural source and/or a modified natural source. Non-dairy analogs are produced to have one or more of the following qualities that are similar or substantially similar to the qualities of comparable dairy products (such as dairy milk or dairy cream): color, taste, nutritional content, stability, dispersibility, and/or solubility. Non-limiting examples of applications of non-dairy analogs is in milk, yogurts, puddings, ice creams, coffee creamers, heavy creams, whipping creams, sour creams, soft cheeses, hard cheeses or other suitable products in which a non-dairy analog may be used. One non-limiting application of non-dairy analogs exemplified in the present disclosure is as a substitute for milk or cream that may be used with tea, coffee, hot chocolate, or other beverages. As described elsewhere herein, in some embodiments at least a portion of the refined protein used in the non-dairy analogs is a deamidated refined protein.

The term “protein concentrate” as used herein refers to the protein material that is obtained from a natural source and/or modified natural source upon removal of at least a portion of (or a substantial portion of) one or more of the following: carbohydrate, ash, and other minor constituents. It typically comprises at least 40% to 70% by weight of protein.

The term “protein isolate” as used herein refers to the protein material that is obtained from a natural source and/or modified natural source upon removal of at least a portion of (or a substantial portion of) one or more of the following: insoluble polysaccharide, soluble carbohydrate, ash, and other minor constituents. It typically has at least 70% by weight of protein.

The terms “refined protein component” or “refined protein” as used herein refers to a protein preparation derived from a natural source and/or modified natural source that contains protein. The term encompasses protein isolate, protein concentrate, flour, meal and/or combinations thereof. In exemplary embodiments of non-dairy analogs or beverage formulations of the present disclosure at least a portion of the refined protein component or refined protein is a deamidated refined protein component or deamidated refined protein.

The term “deamidated protein” or “deamidated refined protein” as used herein refers to a protein preparation modified by in vitro treatment with a glutaminase (e.g., a protein-glutaminase, a peptidoglutaminase II, a peptidylglutaminase II, a glutaminyl-peptide glutaminase). Such modification results in a protein having one or more glutamine residues converted to glutamate residues.

The terms “stable,” “solubilized” and “soluble” as used herein when referring to a protein mixed in a non-dairy analog, beverage formulation, or other aqueous composition (such as coffee or tea), means that the mixture has a uniform, or substantially uniform appearance and may include an insubstantial amount of visible precipitation, or no visible precipitation. It is also to be understood that uniform, or substantially uniform, does contemplate some permitted variation in the color of the mixture or in portions of the mixture.

The term “feathering” as used herein means the presence of particles due at least in part to flocculation or protein aggregation (instability) occurring when the non-dairy analog is dispersed in a hot beverage.

The ingredients of the non-dairy analogs provided herein, such as the refined protein component, may be derived from one or more non-animal natural and/or one or more non-animal modified natural sources. Suitable natural sources are naturally occurring plants, algae, fungi, or microbes.

Examples of suitable plants include, but are not limited to, vegetable plants (e.g., carrot, celery), sunflower, potato, sweet potato, tomato, blueberry, nightshades, buckwheat, amaranth, chard, quinoa, spinach, hazelnut, canola, kale, bok choy, rutabaga, hemp, pumpkin, squash, legume plants (e.g., alfalfa, lentils, beans, clovers, peas, soybean, peanut, chickpea, green pea, yellow pea, snow pea, lima bean, fava bean), cotton, fruiting plants (e.g., apple, apricot, peach, plum, pear, nectarine), strawberry, blackberry, raspberry, cherry, citrus (e.g., grapefruit, lemon, lime, orange, bitter orange, mandarin), mango, grape, broccoli, brussels, sprout, rapeseed (canola), turnip, cabbage, cucumber, watermelon, honeydew melon, zucchini, cassava, baobab, almond, macadamia, taro, barley, corn, oat, palm, rice, rye, sorghum, triticale, moringa, grain plants, leafy vegetables, non-grain legume plants, millets, green algae, derivatives and crosses thereof or combinations thereof. In certain embodiments, examples of suitable plants may be selected from one or more of the following: peas, flaxseed, soybeans, lentils, lupin, fava bean, chickpea, sunflower, rapeseed, sugar cane, sugar beet, oat, wheat and corn. In certain embodiments, the suitable plant may be peas, for example yellow peas. In certain embodiments, the suitable plant may be flaxseed. In certain embodiments, the suitable plant may be soybeans. In certain embodiments, the suitable plant may be lentils. In certain embodiments, the suitable plant may be lupins. In certain embodiments, the suitable plant may be fava beans. In certain embodiments, the suitable plant may be chickpeas. In certain embodiments, the suitable plant may be sunflower. In certain embodiments, the suitable plant may be rapeseed. In certain embodiments, the suitable plant may be sugar cane. In certain embodiments, the suitable plant may be sugar beet. In certain embodiments, the suitable plant may be oat. In certain embodiments, the suitable plant may be wheat. In certain embodiments, the suitable plant may be corn.

Examples of suitable algae include, but are not limited to, viridiplantae, stramenopiles, rhodophyta, chlorophyta, PX, flordeophyceae, bangiophyceae, florideohpyceae, trebouxiophyceae, phaeophyceae, palmariales, gigartinales, bangiales, gigartinales, Chlorella, Laminaria japonica, Laminaria saccharina, Laminaria digitata, Macrocystis pyrifera, Alaria marginata, Ascophyllum nodosum, Ecklonia sp., Palmaria palmata, Gloiopeltis furcata, Porphyra columbina, Gigartina skottsbergii, Gracilaria lichenoides, Chondrus crispus, Gigartina bursa-pastoris, derivatives and crosses thereof or combinations thereof. In certain embodiments, examples of suitable algae may be selected from one or more of the following: Pyropia, Spirolina, rhodophyta, chlorphyta, and chlorella.

Examples of suitable fungi include but are not limited to Pichia pastoris, Saccharomyces cerevisiae, Saccharomyces pombe, derivatives and crosses thereof or combinations thereof. In certain embodiments, examples of suitable fungi may be selected from one or more of the following: Saccharomyces sp., Pichia pastoris, Hansunula polymorpha, Aexula adeninivorans, Kluyveromyces lactis, Yarrowia lipolytica, and Schizosaccaromyces pombe. In certain embodiments, a suitable fungus may be Saccharomyces cerevisiae. Examples of suitable microbes include but are not limited to firmicutes, cyanobacteria (blue-green algae), bacilli, oscillatoriales, bacillales, lactobacillales, oscillatoriales, bacillaceae, lactobacillaceae, arthrospira, Bacillus coagulans, Lactobacillus acidophilus, Lactobacillus Reuteri, Spirulina, Arthrospira platensis, Arthrospira maxima, derivatives and crosses thereof or combinations thereof. In certain embodiments, examples of suitable microbes may be selected from one or more of the following: Escherichia coli, Lactobacillus sp., and Cornybacterium glutamicum. In certain embodiments, a suitable microbe may be a protist, such as Euglena spp.

Non-animal natural sources may be obtained from a variety of sources including, but not limited to, nature (e.g., lakes, oceans, soils, rocks, gardens, forests, plants, animals), brewery stores, and commercial cell banks (e.g., ATCC, collaborative sources). Modified non-animal natural sources may be obtained from a variety of sources including but not limited to brewery stores and commercial cell banks (e.g., ATCC, collaborative sources), or can be generated from natural sources by methods known in the art, including selection, mutation, or gene manipulation. Selection generally involves continuous multiplication and steady increase in dilution rates under selective pressure. Mutation generally involves selection after exposure to mutagenic agents. Gene manipulation generally involves genetic engineering (e.g., gene splicing, insertion of deletions or modifications by homologous recombination) of target genes. A modified natural source may produce a non-native protein, carbohydrate, lipid, or other compound, or produce a non-native amount of a native protein, carbohydrate, lipid, or other compound. In some embodiments, the modified natural source expresses higher or lower levels of a native protein or metabolic pathway compound. In other such embodiments, the modified natural source expresses one or more novel recombinant proteins, RNAs, or metabolic pathway components derived from another plant, algae, microbe, or fungus. In other embodiments, the modified natural source has an increased nutraceutical content compared to its native state. In yet other embodiments, the modified natural source has more favorable growth and production characteristics compared to its native state. In some such embodiments, the modified non-animal natural source has an increased specific growth rate compared to its native state. In other such embodiments, the modified non-animal natural source may utilize a different carbon source than its native state.

In some embodiments, the protein, lipid, carbohydrate, or other ingredients of the non-dairy analogs provided herein are derived from byproducts of previously processed one or more non-animal natural or modified non-animal natural sources. Examples of such byproducts include, but are not limited to, deoiled meal (e.g., deoiled flaxseed meal, deoiled soybean meal, deoiled sunflower meal, deoiled canola meal, or combinations thereof).

Deamidation of Plant Proteins Using Glutaminase

Glutaminase is an enzyme in the class of hydrolytic enzymes that catalyzes the hydrolysis of the γ-amido bond of L-glutamine to L-glutamate and ammonia (Nandakumar et. al., (2003) “Microbial glutaminase: biochemistry, molecular approaches and applications in the food industry,” Journal of Molecular Catalysis B: Enzymatic 23(2003): 87-100). Nearly all living cells produce glutaminase, which plays a significant contributory role in cellular nitrogen metabolism. Glutaminase also has important pharmaceutical and industrial uses as an effective agent in the treatment of acute lymphocytic leukemia and HIV, as an analytical agent, a biosensing agent, as a flavor enhancing agent and in the production of specialty chemicals such as threonine (Sathish & Prakasham (2010) “Enrichment of glutaminase production by Bacillus subtilis RSP-GLU in submerged cultivation based on neural network—genetic algorithm approach,” Journal of Chemical Technology & Biotechnology 85:50-58). Microbial glutaminases have a long history of use and are used extensively in the food industry due to their role as flavor-enhancing agents (see e.g., Sarada (2013) “Production and applications of L-Glutaminase using fermentation technology,” Asia Pacific Journal of Research 1(VIII)).

Glutaminase is also capable of altering protein characteristics and functionality by converting glutamine residues of the protein to glutamate in the process referred to as deamidation. For example, deamidation can alter the secondary and tertiary structure of proteins by converting the amide groups of glutamine and asparagine residues into acidic carboxyl groups with the release of ammonia. This conversion can lead to a decrease in the isoelectric point (pI) of the protein and the resulting deamidated proteins tend to be more soluble under weakly acidic conditions.

A number of different glutaminase enzymes have been used for protein deamidation including transglutaminase, protease, peptide-glutaminase and protein-glutaminase isolated from soil bacteria, e.g., Chryseobacterium proteolyticum. It is contemplated that any glutaminase enzyme known in the art to deamidate proteins can be used in the methods of the present disclosure for preparing non-dairy analog and beverage formulations. Because it is known that different glutaminase enzymes have different activities, different optimal reaction conditions (e.g., pH profiles), and different substrate preferences it is contemplated that one or more different glutaminase enzymes can be used in a process for preparing a deamidated refined protein component for use in a non-dairy analog or beverage formulation. Accordingly, in some embodiments, the present disclosure provides methods wherein the glutaminase is selected from a transglutaminase, protease, peptide-glutaminase, protein-glutaminase, and a combination thereof.

The protein-glutaminase isolated and purified from C. proteolyticum (EC 3.5.1.44) has been shown to be capable of catalyzing deamidation of glutamine residues within low- and high molecular weight proteins, but does not deamidate asparagine residues or free glutamines (see e.g., Yamaguchi et al., (2001) “Protein-glutaminase from Chryseobacterium proteolyticum, an enzyme that deamidates glutamyl residues in proteins. Purification, characterization and gene cloning,” Eur. J. Biochem. 268: 1410-1421). In some embodiments of the methods of the present disclosure, the glutaminase treatment is carried out using a protein-glutaminase purified from C. proteolyticum (EC 3.5.1.44) or otherwise derived or engineered from the protein-glutaminase of C. proteolyticum. In some embodiments, the glutaminase used in the glutaminase treatment of the methods of the present disclosure is Amano PG500, a protein-glutaminase commercially available from Amano Enzyme USA Co., Ltd., Elgin, Ill., USA, as described in the Examples herein.

Methods and conditions useful for the deamidation of plant proteins using protein-glutaminase treatment can vary in accordance with the known conditions useful for enzymatic treatment of proteins. For example, a pea protein isolate may be enzymatically modified with 0.1% to 15% (wt/wt protein) of protein glutaminase to yield deamidated pea protein. Example 1 of the present disclosure provides an exemplary method and conditions for the deamidation of pea protein. The amount of protein glutaminase, or enzyme loading, useful to deamidate a plant protein can vary. In certain embodiments, the enzyme load may be between 0.1% to 15%, 1% to 15%, 0.5% to 10%, 2% to 10%, 3% to 6%, 3% to 10%, 5% to 12%, 6% to 15%, 2% to 4% or 3% to 5% weight protein glutaminase relative to weight pea protein. In certain embodiments, the enzyme load may be approximately 0.1%, 0.5%, 1%, 2%, 2.5%, 3%, 3.5%, 4%, 5%, 6%, 7%, 8%, 9%, 9.5%, 10%, 10.5% 11% or 12% weight protein glutaminase relative to weight plant protein. In certain embodiments, the enzyme load may be at least 0.1%, 0.5%, 1%, 2%, 2.5%, 3%, 3.5%, 4%, 5%, 6%, 7%, 8%, 9%, 9.5%, 10%, 10.5% 11% or up to 15% weight protein glutaminase relative to weight plant protein. In certain embodiments, the plant protein is a pea protein. The incubation time may vary, for example between 0.5 hours to 10 hours, 1 hour to 5 hours, 3 hours to 6 hours, 4 hours to 5 hours or 1 hour to 2.5 hours. In certain embodiments, the incubation time may be between 1 hour to 5 hours. The pH used during incubation or reaction may also vary. For example, the pH may be approximately 6, 7, 8 or 9. The pH may also be between 6 to 9, 7 to 8, 6 to 8 or 7 to 9.

The amount of deamidation of the refined protein may vary depending on the protein and the conditions of the glutaminase treatment. In certain embodiments, the deamidated refined protein resulting from the treatment may be at least 10%, 20%, 30%, 40%, 50%, 60%, 70%, 80%, 90% 95% 98% or 99.9% deamidated. In certain embodiments, the refined protein may be between 40% to 60%, 45% to 55%, 20% to 70%, 30% to 80%, 35% to 90%, 20% to 60% or 40% to 100% deamidated.

Non-Dairy Analogs Containing Deamidated Refined Proteins

Certain embodiments of the present disclosure are directed to a non-dairy analog that comprises a refined protein component obtained substantially from or wholly from non-animal natural products, wherein at least a portion of the refined protein component used in the non-dairy analog is a deamidated refined protein. The non-dairy analogs comprising the deamidated protein has stability, dispersibility, and/or solubility qualities improved relative to non-dairy analogs without deamidated protein and similar in quality to comparable dairy products. It is a surprising effect of the non-dairy analog compositions and formulations of the present disclosure that the inclusion of deamidated refined proteins (e.g., refined pea protein) in non-dairy analogs (e.g., non-dairy milk), improves the stability, dispersibility, and/or solubility of the refined plant protein within acidic beverages such as coffee. In contrast, non-dairy analogs that do not include the deamidated refined protein (e.g., only unmodified refined pea protein isolate) exhibited substantial feathering and/or precipitation when mixed with an acidic beverage such as coffee. Deamidated refined proteins (e.g., pea protein) when included within the same non-dairy analog are stable, do not feather, and/or precipitate when mixed with an acidic beverage such as coffee.

In certain embodiments, the non-dairy analog exhibits less than 5%, 3%, 1% or 0.5% by volume precipitation when added to the aqueous composition after at least partially mixing of the non-dairy analog with the aqueous composition, wherein the aqueous composition is between 30° C. and 95° C. and has an aqueous composition pH that is less than 7, before the non-dairy analog is combined with the aqueous composition or after the non-dairy analog is combined with the aqueous composition.

In certain embodiments, the non-dairy analog is solubilized, or substantially solubilized, in the aqueous composition 15 minutes, 10 minutes, or 5 minutes after at least partially mixing of the non-dairy analog composition with the aqueous composition.

In certain embodiments, the non-dairy analog exhibits less than 5%, 3%, 1% or 0.5% by volume precipitation when added to the aqueous composition after at least partially mixing of the non-dairy analog with the aqueous composition 15 minutes, 10 minutes, or 5 minutes after at least partially mixing of the non-dairy analog composition with the aqueous composition.

In certain embodiments, the non-dairy analog does not visibly precipitate when added to the aqueous composition for 15 minutes, 10 minutes, or 5 minutes after at least partially mixing of the non-dairy analog composition with the aqueous composition.

In certain embodiments, the non-dairy analog exhibits insubstantial precipitation when added to the aqueous composition for 15 minutes, 10 minutes, or 5 minutes after at least partially mixing of the non-dairy analog composition with the aqueous composition.

In certain embodiments, the non-dairy analog exhibits less than 5%, 3%, 1% or 0.5% by volume precipitation when added to the aqueous composition after at least partially mixing of the non-dairy analog with the aqueous composition.

In certain embodiments, the non-dairy analog does not exhibit visible feathering when added to the aqueous composition for 15 minutes, 10 minutes, or 5 minutes after at least partially mixing of the non-dairy analog with the aqueous composition.

In some embodiments, the non-dairy analogs provided herein are analogs of dairy milk. In other embodiments, the non-dairy analogs are analogs of dairy cream type products derived from dairy milk. In some embodiments, the non-dairy analogs are primarily, substantially, or entirely composed of ingredients derived from non-animal natural sources. In alternative embodiments, the non-dairy analogs are composed of ingredients partially derived from animal sources but supplemented with ingredients derived from non-animal natural sources.

In certain embodiments, the amount of refined protein used in the non-dairy analog may vary. In certain embodiments, the refined protein component is at 0.2%, 0.5%, 1%, 2%, 3%, 5%, 8%, 10%, 15%, or 20% by weight of the total weight of the non-dairy analog. In certain embodiments, the refined protein component is between 0.2% to 10%, 0.5% to 5%, 1% to 10%, 2% to 5%, 3% to 4%, 3% to 8%, 2% to 4%, 5% and 15% or 10% and 16% by weight of the total weight of the non-dairy analog. In some embodiments, the ratio of protein to lipid in the non-dairy analogs is 1:5, 1:4, 1:3, 1:2, 1:1, 2:1, 3:1, 4:1, 5:1, or 10:1. Protein content of a food product may be determined by a variety of methods, including, but not limited to, AOAC International reference methods AOAC 990.03 and AOAC 992.15, and combustion analysis (ISO 14891:2008).

In the non-dairy analog embodiments disclosed herein, the amount of deamidated refined protein used in the refined protein component may vary. In certain embodiments, the deamidated refined protein component is at least 10%, 20%, 30%, 40%, 50%, 70%, 80%, 90% or 100% by weight of the total weight of the refined protein component used in the non-dairy analog. In certain embodiments, the deamidated refined protein component is between 10% and 100%, 20% to 60%, 30% to 50%, 40% to 70%, 50% to 80%, 70% to 90% or 35% to 45% by weight of the total weight of the refined protein component used in the non-dairy analog.

As noted elsewhere herein, various methods and conditions can be used for generating deamidated plant proteins, and one exemplary method for pea protein is provided in Example 1 below. The resulting amount of deamidation of the refined protein relative to the amount non-deamidated refined protein may be varied. In certain embodiments, the refined protein may be at least 10%, 20%, 30%, 40%, 50%, 60%, 70%, 80%, 90% 95% 98% or 99.9% deamidated. Accordingly, in certain embodiments of the non-dairy analog, the refined protein may be between 40% to 60%, 45% to 55%, 20% to 70%, 30% to 80%, 35% to 90%, 20% to 60% or 40% to 100% deamidated.

The non-dairy analogs provided herein may further comprise lipids. In some embodiments, the dairy product analogs comprise between 1% and 10%, between 0.5% and 8%, between 1% and 7%, between 5% and 20%, between 10% and 25% or between 5% and 10% by weight of lipids obtained from non-animal natural sources. Lipid content of a food product may be determined by a variety of methods, including, but not limited to, AOAC International reference method AOAC 954.02. Examples of suitable lipids include, but are not limited to, almond oil, aloe vera oil, apricot kernel oil, avocado oil, baobab oil, calendula oil, canola oil, coconut oil, corn oil, cottonseed oil, evening primrose oil, grape oil, grape seed oil, hazelnut oil, jojoba oil, linseed oil, macadamia oil, natural oils, neem oil, non-hydrogenated oils, olive oil, palm oil, partially hydrogenated oils, peanut oil, rapeseed oil, sesame oil, soybean oil, sunflower oil, synthetic oils, vegetable oil, omega-fatty acids (e.g., arachidonic acid, omega-3-fatty acids, omega-6-fatty acids, omega-7-fatty acids, omega-9-fatty acids), or combinations thereof. In certain embodiments, examples of suitable lipids may be selected from one or more of the following: sunflower oil, coconut oil, sunflower lecithin, palm oil or combinations thereof. In certain embodiments, the lipid may be sunflower oil. In certain embodiments, the lipid may be sunflower lecithin. In certain embodiments, the lipid may be palm oil. In certain embodiments, the lipid may be coconut oil. In certain embodiments, the lipid may be soy lecithin.

In some embodiments, the non-dairy analogs provided herein comprise similar, substantially similar, or reduced amounts of carbohydrate as analogous dairy products. Carbohydrate content of a food product may be determined by a variety of methods, including, but not limited to, high performance liquid chromatography. Examples of suitable carbohydrates include, but are not limited to, sucrose, glucose, fructose, mannose, steviosides, artificial sweeteners, monk fruit extract or combinations thereof. In certain embodiments, examples of suitable carbohydrates may be selected from one or more of the following: sucrose, glucose, and fructose. In certain embodiments, the carbohydrate may be monk fruit extract. In certain embodiments, the carbohydrate may be sucrose. In certain embodiments, the carbohydrate may be fructose. In certain embodiments, the carbohydrate may be artificial sweeteners. In some embodiments, the non-dairy analogs comprise between 0.5% and 15%, between 1% and 10%, or between 3% and 8% by weight of carbohydrate. In some embodiments, the dairy product analog comprises at least 0.5%, 1%, 3%, 5%, 8% 10% or 15% by weight of carbohydrate. In some embodiments, the non-dairy analog comprises 30%, 40%, 50%, 60%, or 70% by weight less total carbohydrate than in an equivalent sized serving of non-dairy analog, regardless of fat content. In some embodiments, the non-dairy analogs do not comprise lactose. In some embodiments, the non-dairy analog contains less than 5%, 3%, 1%, or 0.5% by weight of lactose. In some embodiments, the non-dairy analog comprises sucrose.

Various protein sources may be used in one or more of the disclosed embodiments. Examples of protein sources include, but are not limited to, melon, barley, coconut, rice, pear, emmer, carrot, lupin seeds, pea, fennel, lettuce, oat, cabbage, celery, soybeans, almond, rice, flax, potato, sunflower, mushroom, or combinations thereof. Other suitable plants and/or protein sources may also be used.

The protein of the plant may be derived from a legume. Examples of legumes include, but are not limited to, alfalfa, lentils, beans, clovers, peas, fava coceira, frijole bola roja, frijole negro, lespedeza, licorice, lupin, mesquite, carob, soybean, peanut, tamarind, wisteria, cassia, chickpea, garbanzo, fenugreek, green pea, yellow pea, snow pea, lima bean, fava bean, black bean, baby bean or combinations thereof. In certain embodiments, the legumes may be selected from peas. In certain embodiments, the legume may be yellow pea. In certain embodiments, the legume may be green pea. In certain embodiments, the legume may be lentils. In certain embodiments, the legume may be chickpeas. In certain embodiments, the legume may be lupin. In certain embodiments, the legume may be fava beans.

Flavorings may also be used in certain embodiments of the non-dairy analogs disclosed herein. Examples, of flavorings include, but are not limited to, chocolate, toffee, almond, truffles, cinnamon, eggnog, caramel, sugar, butter pecan, hazelnut, pumpkin spice, peppermint, coconut, French vanilla or combinations thereof. In certain embodiments, natural sweetness enhancers may be used.

Beverage Formulations Containing Deamidated Refined Protein

As described above, non-dairy analogs containing a deamidated refined protein component exhibit improved qualities of improved dispersibility and/or solubility, and decreased visible precipitation of the protein component when mixed in an aqueous beverage such as coffee or tea. These improved qualities also extend to the use of a deamidated refined protein component in other protein-containing beverage formulations. A wide range of beverage formulations incorporate a significant portion of a refined protein component in an aqueous mixture. Such protein-supplemented beverage formulations include but are not limited to, protein drinks, post-work-out drinks, vitamin drinks, exercise drinks, electrolyte drinks, fruit juice drinks, and iced tea drinks. It is contemplated that the beverage formulations comprising a deamidated refined protein component can be used for any aqueous beverage that is supplemented with a plant protein. Generally, it is highly desirable to minimize any precipitation of a refined protein component in such beverages because consumers generally prefer a substantially transparent beverage without gritty or chalky solids present.

Accordingly, in some embodiments, the present disclosure provides a beverage formulation, wherein the formulation comprises: (a) a refined protein component in which at least a portion of the refined protein component is a deamidated refined protein component; (b) water or carbonated water; and (c) a pH of between 6 and 9.

In some embodiments of the beverage formulation, the refined protein component is sourced from a plant; optionally, sourced from a legume. In some embodiments, the refined protein component is sourced from a pea plant or a pea protein. Generally, the wide range of plant proteins described elsewhere herein as useful with non-dairy analogs can also be used as a refined protein component in the beverage formulations of the present disclosure.

In some embodiments the beverage formulation can comprise additional ingredients selected from: (a) sugar and/or a carbohydrate; (b) at least one vitamin or mineral; (c) at least one lipid from a non-animal natural source; and/or (d) at least one emulsifier, and/or a hydrocolloid or gum. Generally, the ingredients described elsewhere herein as useful with non-dairy analogs (e.g., carbohydrates, emulsifiers, lipids, etc.) can be used as ingredients in the beverage formulations of the present disclosure.

As described elsewhere herein for non-dairy analogs, it is an advantage of the beverage formulations of the present disclosure that the deamidated portion of the refined protein component (e.g., using treatment with 0.02-0.1 g glutaminase per g protein) greatly improves the protein solubility, dispersibility, and/or other precipitation-related qualities of the beverage. In some embodiments the improved protein precipitation-related qualities of the beverage formulation: (a) exhibits less than 5%, 3%, 1% or 0.5% by weight precipitation of the refined protein component; and/or (b) exhibits less than 5%, 3%, 1% or 0.5% by volume precipitation of the refined protein component.

These improved protein solubility and/or dispersibility qualities of the beverage formulations of the present disclosure are increasingly important in beverage formulation comprising substantial amount of protein. In some embodiments of the beverage formulations it is contemplated that the refined protein component comprises is at least 1%, 2%, 3%, 5%, 8% or 10% by weight of the beverage formulation.

As described with respect non-dairy analogs, the amount of deamidated refined protein used in a beverage formulation can be varied depending on the particular beverage application. For example, a high-protein post-workout beverage formulation may require a higher level of deamidated refined protein component in order to keep the overall protein solubility and/or dispersibility at desired levels. Accordingly, in some embodiments of the beverage formulation, the deamidated refined protein component is at least 10%, 20%, 30%, 40%, 50%, 60%, 70%, 80%, 90% or 100% by weight of the total weight of the refined protein component.

Methods for Preparing Non-Dairy Analogs and Beverage Formulations

In certain embodiments, the present disclosure provides compositions, formulations, and methods for producing the non-dairy analogs and beverage formulations that contain a deamidated refined protein component. General methods, compositions, and formulations useful for preparing the non-dairy analogs and beverage formulations of the present disclosure are known in the art. See e.g., WO2017/120597A1, published Jul. 13, 2017, which is hereby incorporated by reference herein. Additional compositions, formulations, and methods of preparation are described elsewhere herein. See e.g., Tables 3 and 4 of Examples 4 and 5, which describe compositions and ranges of ingredients useful in the non-dairy analogs and beverage formulations of the present disclosure.

In some embodiments, the methods are for producing the non-dairy analog may comprise one or more of the following steps, in or out of order:

a) obtaining at least one lipid from a non-animal natural source;

b) obtaining at least one refined protein component from a non-animal natural source in which at least 10% by weight of the refined protein component is a deamidated refined protein component;

c) blending the at least one lipid and the at least one refined protein component with water to generate a mixture; and

d) emulsifying at least a portion of the mixture to provide a non-dairy analog;

whereby the quantities and proportions of the at least one lipid, and the at least one refined protein components are selected so as to provide a desired stability, dispersibility, and/or solubility and the non-dairy analog has a pH of between 4.0 and 10. In some embodiments, the non-dairy analog can have a pH of between 6.5 and 10.

In some embodiments, the methods are for producing the non-dairy analog may comprise one or more of the following steps, in or out of order:

a) obtaining at least one lipid from a non-animal natural source;

b) obtaining at least one refined protein component from a non-animal natural source in which at least a portion of the refined protein component is a deamidated refined protein component;

c) blending the at least one lipid and the at least one refined protein component with water to generate a mixture; and

d) emulsifying at least a portion of the mixture to provide a non-dairy analog;

whereby the quantities and proportions of the at least one lipid, and the at least one refined protein components are selected so as to provide a desired stability, dispersibility, and/or solubility and the non-dairy analog.

In some embodiments, the methods are for producing the beverage formulation of the present disclosure include one or more of the following steps, in or out of order:
a) providing at least one refined protein component from a non-animal natural source in which at least 10% by weight of the refined protein component is a deamidated refined protein component;
b) providing at least one other ingredient selected from: (i) sugar and/or a carbohydrate; (ii) a vitamin or a mineral; (iii) a lipid from a non-animal natural source; (iv) an emulsifier; and/or (v) a hydrocolloid or a gum.
c) providing water or carbonated water; and
d) blending the at least one refined protein component and the at least one other ingredient with water to generate a mixture.

In preparing the beverage formulation, the quantities and proportions of the at least one refined protein component, including the amount of deamidated protein component, the other ingredients, and the water or carbonated water, can be selected so as to provide a desired stability, dispersibility, and/or solubility a pH of between 6 and 9.

In any of the methods for producing a non-dairy analog or a beverage formulation comprising a deamidated refined protein component, it is contemplated that the method can be carried out wherein an unmodified refined protein component is provided during an initial step of the process and a subsequent step of incubation with glutaminase is carried out whereby at least a portion of the unmodified refined protein component deamidated in situ, that is, deamidation occurs during the process of preparing the non-dairy analog or beverage formulation. Accordingly, in some embodiments, the present disclosure provides a method for preparing a non-dairy analog, the method comprising:

(a) generating an aqueous mixture at pH 7.5-8.5 and 35-60° C. comprising: an 0.5-5.0 wt % unmodified refined protein component from a non-animal natural source, and 0.3-1.0 wt % buffering salts;

(b) adding to the mixture 0.1-1.0 wt % of a glutaminase and mixing at 35-60° C. for at least 1 hour, whereby at least a portion of the unmodified refined protein component is deamidated;

(c) after mixing of step (b) adding to the mixture 2-10 wt % oil, 0.01-0.05 wt % gums, 0-1 wt % emulsifier, 0-1 wt % flavors and 0-2 wt % sugar; and

(d) heating the mixture to 90° C. and subjecting to homogenization.

As with the other methods disclosed herein, the unmodified refined protein component can be sourced from a plant, optionally, sourced from a legume, and in some embodiments, the unmodified refined protein component is sourced from a pea plant or a pea protein. Also, as in the other methods involving the use of deamidated proteins prepared by glutaminase treatment, the in situ deamidation process can be carried out using a glutaminase that is a protein glutaminase, and in some embodiments, the protein-glutaminase is isolated or derived from C. proteolyticum. As noted elsewhere herein, it is contemplated that glutaminases such as protein-glutaminase useful in the methods of the present disclosure can include a non-naturally occurring protein-glutaminase, such as a protein-glutaminase produced by engineering (e.g., by site-directed mutagenesis) the naturally-occurring protein-glutaminase from C. proteolyticum. Techniques, such as directed-evolution, are well-known in the art for engineering enzymes with improved properties (e.g., increased activity and/or selectivity, heat stability, high-pH or low-pH stability) for use in industrial processes. Accordingly, in some embodiments of the in situ deamidation process for preparing a non-dairy analog, the steps (a) and (b) are carried out at a temperature of 35-60° C., 40-55° C., or 45-50° C. Similarly, depending on the particular conditions and glutaminase used it is contemplated that the step (b) mixing time can be varied from 1 to 12 hours, from 1 to 6 hours, or from 1 to 3 hours. It is also contemplated that the buffering salts used in the in situ deamidation can be varied depending on particular conditions and glutaminase used. In some embodiments, the buffering salts can comprise chloride salts (e.g., sodium or potassium) and/or phosphate salts (e.g. sodium or potassium), and optionally, in some embodiments, the buffering salts are phosphate salts.

Methods for obtaining the at least one lipid from a non-animal natural are known in the art. Methods for obtaining the at least one refined protein component from a non-animal natural and/or modified non-animal natural source are provided herein. Other methods for obtaining the at least one refined protein component are known in the art. Methods for obtaining the deamidated refined protein component from a non-animal natural and/or modified non-animal natural source are provided herein or are known in the art. Other methods for obtaining the deamidated refined protein component are known in the art. In some embodiments, the lipid and/or refined protein component are obtained as slurries. In some embodiments, the lipid and/or refined protein component are obtained in solid form. In some embodiments, the refined protein component is combined with one or more other proteins prior to being mixed with the at least one lipid.

The at least one refined protein component may be added to the water as a dry, or substantially dry, solid or as a slurry. In certain embodiments, the at least one refined protein component as a dry, or substantially dry, solid may contain at least 50%, 60%, 70%, 80%, 90% by weight protein. In certain embodiments, the at least one refined protein component as a dry, or substantially dry, solid may contain between 50% to 100%, 70% to 90% or 80% to 100% by weight protein. In certain embodiments, the at least one refined protein component as a slurry may contain at least 3%, 5%, 10%, 20%, 30% or 40% by weight protein. In certain embodiments, the at least one refined protein component as a slurry may contain between 3% to 40%, 5% to 30%, 5% to 20% or 10% to 30% by weight protein.

The water or aqueous component may be an aqueous liquid, including but not limited to pure water, tap water, bottled water, deionized water, spring water, or a mixture thereof. The aqueous component may also contain suitable dissolved materials.

The lipid, protein, and aqueous components may be mixed in various orders. In some embodiments, the three components are mixed simultaneously. In other embodiments, the lipid is mixed with the protein component before the aqueous component is introduced into the mixture. In yet other embodiments, the protein component is mixed with the aqueous component before the lipid is introduced into the mixture. In yet other embodiments, the lipid is mixed with the aqueous component before the protein component is introduced into the mixture.

Combining the lipid, protein, and aqueous components may be accomplished using a variety of mixing devices, for example, mechanical agitators and/or pressure jets. The components may also be stirred or mixed by hand. Mixing may continue until the components are distributed substantially evenly throughout the mixture.

In some embodiments, a carbohydrate component may be also added. A variety of ingredients may be used as the carbohydrate component, including but not limited to starch, simple sugars, flour, edible fiber, and combinations thereof. Examples of suitable starches include but are not limited to maltodextrin, inulin, fructo oligosaccharides, pectin, gum Arabic, carboxymethyl cellulose, guar gum, gellan gum, corn starch, oat starch, potato starch, rice starch, wheat starch, or combinations thereof. Examples of suitable flour include but are not limited to amaranth flour, oat flour, quinoa flour, rice flour, rye flour, sorghum flour, soy flour, wheat flour, corn flour, or combinations thereof. Examples of suitable edible fiber include but are not limited to barley bran, carrot fiber, citrus fiber, corn bran, soluble dietary fiber, insoluble dietary fiber, oat bran, pea fiber, rice bran, head husks, soy fiber, soy polysaccharide, wheat bran, wood pulp cellulose, or combinations thereof. In some embodiments, the carbohydrate component may be guar gum. In some embodiments, a carbohydrate component may be gellan gum. In some embodiments, a carbohydrate component may be a polysaccharide. In some embodiments, the carbohydrate component does not comprise lactose or substantially does not comprise lactose. The carbohydrate component may be present in the aqueous component before mixing. Alternatively, the carbohydrate component is added to the lipid and/or protein components or to the lipid, protein, and aqueous mixture.

Thickening agents may be used, including gelatin, pectin, agar, gums, starches, and ultra-gel. Examples of acceptable gums include sodium alginate, gellan gum, xanthan gum, guar gum or combinations thereof. Examples of acceptable starches include tapioca starch, arrowroot starch or combinations thereof. In some embodiments, the thickening agent may be a guar gum. In some embodiments, the thickening agent may be a gellan gum.

In some embodiments, one or more other ingredients are further added. In some such embodiments, the one or more other ingredients are added to the aqueous component before mixing. In other embodiments, the one or more other ingredients are added to the lipid and/or protein components or to the lipid, protein, and aqueous mixture. In some embodiments, the one or more other ingredients include calcium.

Emulsification may occur without additional mechanical energy, or require mechanical energy (for example, vortexing, homogenization, agitation, sonication, or other suitable mechanical activity). When emulsification is aided by lower amounts of mechanical energy (for example, agitation in a conventional mixer under moderate shear of between about 100 rpm and about 1,000 rpm), the average droplet size of the resulting emulsion is typically larger (for example, at least about 75% of the droplets have a diameter greater than about 25 um). When emulsification is aided by higher amounts of mechanical energy (e.g., homogenization in a high-pressure [for example, between about 35 bar and about 650 bar] 1- or 2-stage homogenizer [e.g., between about 1,000 psi and about 10,000 psi], or microfluidic homogenization [between about 500 and about 2,000 bar], the average droplet size of the resulting emulsion is typically smaller (for example, at least about 75% of the droplets have a diameter of less than about 10 um). Nanoemulsions may be obtained by homogenizing in a microfluidizer or other suitable equipment. In certain applications, to obtain higher lipid emulsions, the lipid component may be added gradually during mixing. Heating may aid in emulsification in certain applications. In some embodiments, emulsification is performed at greater than room temperature, greater than 30° C., 40° C., 50° C., 60° C., 70° C., or 80° C., between 90° C. and 120° C., between 30° C. and 60° C., or between 40° C. and 50° C. Heating is generally followed by cooling. Emulsification may be monitored by removing a sample of the mixture and analyzing it by such methods as microscopy, light scattering, and/or refractometry.

The emulsions may have droplets of various sizes. In some embodiments, the emulsions are polydisperse emulsions (i.e., emulsions comprising droplets with a broad distribution of droplet sizes). In other embodiments, the emulsions are monodisperse (i.e., emulsions comprising droplets with a narrow distribution of droplet sizes). In some embodiments, the emulsions are microemulsions (i.e., thermodynamic stable systems of dispersed droplets in continuous phase). In other embodiments, the emulsions are nanoemulsions (i.e., metastable [or kinetically stable] dispersions of one liquid in different immiscible liquid having droplet sizes ranging from 1 to 100 nm). In some embodiments, the emulsions have an average droplet size of less than about 1,000 nm, less than about 750 nm, less than about 500 nm, less than about 250 nm, less than about 100 nm, or less than about 50 nm, between about 100 nm and about 800 nm, or between about 100 nm and about 300 nm. In some embodiments, droplet sizes are reduced to reduce the lipid contents of the emulsions and non-dairy analogs provided herein. The degree of emulsification achieved and hence the final textures of the emulsions may be controlled to a certain degree by varying certain parameters during emulsification. Examples of such parameters include, but are not limited to, the type and/or amount of lipid component, the type and/or amount of protein component, the type and/or amount of optional emulsifiers, the amount of mechanical energy used during emulsification, the centrifugation or filtration techniques, the pH of the aqueous component, the temperature during mixing, the amount of optional salt in the aqueous component or combinations thereof.

In some embodiments, the non-dairy analog may be sterilized or pasteurized. Sterilization may occur by UV irradiation, heating (e.g. steam sterilization, flaming, or dry heating), or chemical sterilization (e.g., exposure to ozone). In some embodiments, sterilization kills more than 95% of microbes. For pasteurization, the non-dairy analog may be heated to a temperature (e.g., between about 280 and about 306° F.) and held at such temperature for a period of time (e.g., between about 1 and about 10 seconds). Appropriate pasteurization steps are known in the art of food manufacturing and may be undertaken at a variety of temperatures and/or for a variety of time durations. Pasteurization may be high-temperature, short-time (HTST), “extended shelf life” (ESL) treatment, high pressure pasteurization (HPP), ultra-pasteurization (UP), ultra-high temperature (UHT) or combinations thereof. A controlled chilling system may be used to rapidly cool the non-dairy analog. In some embodiments, the non-dairy analogs undergo vacuum cooling to remove volatiles and water vapor following pasteurization.

The non-dairy analog may optionally be dried to obtain powders. Drying may be performed in a suitable way, including but not limited to spray drying, dry mixing, agglomerating, freeze drying, microwave drying, drying with ethanol, evaporation, refractory window dehydration or combinations thereof.

Refined Protein Components

In some embodiments, the refined protein component has a total protein content of at least 30%, 40%, 50%, 60%, 70% or 80% by dry weight.

In some embodiments, the refined protein component has a total protein content of between 30% and 90%, between 40% and 85%, between 50% and 90%, between 65% and 88%, between 70% and 86%, or between 75% and 86% by dry weight.

In some embodiments, the refined protein component does not include any added calcium. It is contemplated, however, the that in some embodiments that the refined protein component has a total bound calcium content of at least 0.05%, 0.1%, 0.3%, 0.5%, 1%, 1.5%, 1.7% or 2% by dry weight.

In some embodiments, the refined protein component has a total bound calcium content of between 0 and 2%, between 0.1% and 2%, between 0.3% and 1.7%, between 0.5% and 1.5%, or between 0.5% and 1% by dry weight.

In some embodiments, the refined protein component is a paste comprising between 4% and 25% by weight of protein, between 0 and 2% by weight of calcium, and between 50% and 92% by weight of water. In some embodiments, the refined protein component is a dry powder comprising between 70% and 90% by weight of protein, and between 0.1% and 2% by weight of calcium.

One exemplary refined protein component has a composition of at least about 80% of visible protein bands on a denaturing protein gel with a molecular weight of less than 200 kDa, at least about 80% of visible protein bands on a denaturing protein gel with a molecular weight of less than 150 kDa on a denaturing protein gel, at least about 80% of visible protein bands on a denaturing protein gel with a molecular weight of between about 10 kDa and about 100 kDa.

Certain embodiments are directed to a refined protein (isolate and/or component) that may have one or more of the following characteristics:

A refined protein comprising between 5% to 97%, 20% to 90%, 30% to 85%, or 40% to 80%, by weight of a protein obtained from one or more non-animal natural sources. A refined protein comprising at least 5%, 10%, 20%, 30%, 40%, 50%, 60%, 70%, 80%, 90% by weight of a protein obtained from one or more non-animal natural sources.

In certain embodiments, the refined protein may be a paste, a wet suspension or a dry powder.

In certain embodiments, the refined protein may have a dry solids weight percentage of at least 5%, 10%, 20%, 30%, 40%, 50%, 60%, 70%, 80%, or 90%.

In certain embodiments, the refined protein may have a calcium to protein ratio is between 0% w/w and 2% w/w, 0.1% w/w to 2% w/w, 0.5% w/w to 5% w/w, 1% w/w to 5% w/w, 3% w/w to 8% w/w, or 5% w/w to 10% w/w.

In certain embodiments, the refined protein may be color neutral or not color neutral.

In certain embodiments, the refined protein may have a pH of between 3 and 11, 6.5 and 10, 5.5 and 8, or 5.7 to 6.7. In certain embodiments, the refined protein may have a pH of at least 3. In certain embodiments, the refined protein may have a pH of less than 9.

In certain embodiments, the refined protein may have a moisture content of between 3% and 90% by weight. In certain embodiments, the refined protein may have a moisture content of at least 3% by weight. In certain embodiments, the refined protein has a moisture content of less than 80% by weight.

In certain embodiments, the refined protein may have a fat content of between 1% and 30% by weight. In certain embodiments, the refined protein may have a fat content of at least 1% by weight. In certain embodiments, the refined protein may have a fat content of less than 25% by weight.

In certain embodiments, the refined protein may have a carbohydrate content of between 0% and 50% by weight. In certain embodiments, the refined protein may have a carbohydrate content of at least 0% by weight. In certain embodiments, the refined protein may have a carbohydrate content of less than 25% by weight.

In certain embodiments, the refined protein has a starch content of between 0% and 10% by weight. In certain embodiments, the refined protein has a starch content of at least 2% by weight. In certain embodiments, the refined protein has a starch content of less than 9% by weight.

In certain embodiments, the refined protein has a phosphorus content of between 0% and 6% by weight. In certain embodiments, the refined protein has a phosphorus content of at least 0.1% by weight. In certain embodiments, the refined protein has a phosphorus content of less than 4% by weight.

In certain embodiments, the refined protein has sodium and/or potassium content of less than 0.5% by weight.

In certain embodiments, the refined protein has an ash content of between 0% and 20% by weight. In certain embodiments, the refined protein has an ash content of at least 1% by weight. In certain embodiments, the refined protein has an ash content of less than 10% by weight.

In certain embodiments, the refined protein has a reducing capacity of between 5% and 50%. In certain embodiments, the refined protein has a reducing capacity of at least 6%. In certain embodiments, the refined protein has a reducing capacity of less than 46%.

In certain embodiments, the refined protein has a total HPLC peak area for total extractable soluble sugars and organic acids of between 20,000 and 250,000. In certain embodiments, the refined protein has a total extractable soluble sugars and organic acids of at least 22,000. In certain embodiments, the refined protein has a total extractable soluble sugars and organic acids of less than 240,000.

In certain embodiments, the refined protein has a total peak area measured by GC analysis of volatile compounds component of between 50,000 and 3,000,000. In certain embodiments, the refined protein has a volatile compounds component of less than 2,500,000.

In certain embodiments, the refined protein has an isoflavones component of between 0% and 0.1% of dry mass. In certain embodiments, the refined protein has an isoflavones component of less than 0.075% of dry mass.

In certain embodiments, the refined protein has a tannins component of between 0% and 0.5% of dry mass. In certain embodiments, the refined protein has a tannins component of less than 0.3% of dry mass.

In certain embodiments, the refined protein has an instability index of between 0.2 and 0.6. In certain embodiments, the refined protein has an instability index of at least 0.22. In certain embodiments, the refined protein has an instability index of less than 0.57.

In certain embodiments, the refined protein has been produced in quantities of at least between 500-kg and 3000-kg, between 200-g and 1000-kg, between 1000-kg and −2500-kg and between 1000-kg and 3500-kg.

Methods for Obtaining Refined Protein Components

Certain embodiments are directed to methods for obtaining refined protein components from non-animal natural sources. Some of the advantages of the methods provided herein is that they may remove, or substantially remove, flavoring agents, aroma agents, coloring agents, other agents or combinations thereof from refined protein preparations, and thus make the refined protein preparations more suitable for use in non-dairy analog. Removal of such agents may also increase the shelf life of non-dairy analogs comprising such refined protein components.

The methods provided herein for obtaining refined protein components from non-animal natural sources may comprise one or more of the following steps, in or out of order:

a. obtaining a protein preparation from a non-animal natural source;

b. washing the protein preparation at a wash pH;

c. extracting the protein preparation at an extraction pH to obtain an aqueous protein solution;

d. separating the aqueous protein solution from non-aqueous components;

e. adding salt;

f. precipitating the protein from the aqueous protein solution at a precipitation pH to obtain a protein precipitate;

g. separating the protein precipitate from non-precipitated components; and

h. washing the protein precipitate to obtain a refined protein component.

The refined protein preparation obtained from a natural source may have various forms, including, but not limited to, protein concentrate, protein isolate, flour, protein meal; native, denatured, or renatured protein; dried, spray dried, or not dried protein; enzymatically treated or untreated protein; and combinations thereof. It may consist of particles of one or more sizes, and may be pure or mixed with other components (e.g., other plant source components). The refined protein preparation may be derived from non-animal natural sources, or from multiple natural sources. In some embodiments, the refined protein preparation is obtained from a plant. In some such embodiments, the plant is legume. In some such embodiments, the legume is pea. The pea may be whole pea or a component of pea, standard pea (i.e., non-genetically modified pea), commoditized pea, genetically modified pea, or combinations thereof. In some embodiments, the pea is Pisum sativum. In some embodiments, the legume is soy. The soy may be whole soy or a component of soy, standard soy (i.e., non-genetically modified soy), commoditized soy, genetically modified soy, or combinations thereof. In some embodiments, the legume is chickpea. The chickpea may be whole chickpea or a component of chickpea, standard chickpea (i.e., non-genetically modified chickpea), commoditized chickpea, genetically modified chickpea, or combinations thereof. In some embodiments, the refined protein preparation may be pre-treated for various purposes, such as, for example, extracting the protein preparation in a solvent to remove lipids, and heat treating the protein preparation to remove volatiles.

Washing the refined protein preparation may utilize various methods, including single wash, multiple washes, and/or counter-current washes.

The wash and extraction pH may be a pH that is suitable for washing and solubilizing proteins in a protein preparation. A suitable wash and extraction pH may be determined by testing various pH conditions, and identifying the pH condition at which the most optimal yield and quality (judged by, for example by one or more of the following: flavor, odor, color, nitrogen content, Ca content, heavy metal content, emulsification activity, MW distribution, and thermal properties of the protein component obtained) of the refined protein component is obtained. In some embodiments, the wash and extraction pH are alkaline pH. In some such embodiments, the alkaline pH is at least 7.1, at least 8, at least 9, at least 10, at least 11, at least 12, between 7.1 and 10, between 8 and 10, between 9 and 10, or between 8 and 9. In some such embodiments, the alkaline pH is 8.5. In some embodiments, the wash and extraction pH are acidic pH. In some such embodiments, the acidic pH is less than 7, less than 6.95, less than 6.5, less than 5, less than 4, less than 3, between 2 and 6.95, between 3 and 6, or between 3 and 5. The extraction pH may be adjusted using a pH adjusting agent. In some embodiments, the pH adjusting agent is a food grade basic pH adjusting agent. In other embodiments, the pH adjusting agent is a food grade acidic pH adjusting agents. Examples of suitable acidic pH adjusting agents include, but are not limited to, phosphoric acid, acetic acid, hydrochloric acid, citric acid, succinic acid, and combinations thereof. Examples of suitable basic pH adjusting agents include, but are not limited to, potassium bicarbonate, sodium bicarbonate, sodium hydroxide, potassium hydroxide, calcium hydroxide, ethanolamine, calcium bicarbonate, calcium hydroxide, ferrous hydroxide, lime, calcium carbonate, trisodium phosphate, and combinations thereof. It is useful to obtain substantially as much extracted protein as is practicable so as to provide an overall high product yield. The yield of protein in the aqueous protein solution may vary widely, wherein typical yields range from 1% to 90%. The aqueous protein solution typically has a protein concentration of between 1 g/L and 300 g/L. The molecular weight distribution of the proteins comprised in the aqueous protein solution may vary widely.

Separating the aqueous protein solution from non-aqueous components may be accomplished by various methods, including but not limited to, centrifugation followed by decanting of the supernatant above the pellet, or centrifugation in a decanter centrifuge. The centrifugation may be followed by disc centrifugation and/or filtration (e.g., using activated carbon) to remove residual protein source material and/or other impurities. The separation step may be conducted at various temperatures within the range of 1° C. to 100° C. For example, the separation step may be conducted between 10° C. and 80° C., between 15° C. and 70° C., between 20° C. and 60° C., or between 25° C. and 45° C. The non-aqueous components may be re-extracted with fresh solute at the extraction pH, and the protein obtained upon clarification combined with the initial protein solution for further processing as described herein. The separated aqueous protein solution may be diluted or concentrated prior to further processing. Dilution is usually affected using water, although other diluents may be used. Concentration may be affected by membrane-based methods. In some embodiments, the diluted or concentrated aqueous protein solution comprises between 1 g/L and 300 g/L, between 5 g/L and 250 g/L, between 10 g/L and 200 g/L, between 15 g/L and 150 g/L, between 20 g/L and 100 g/L, or between 30 g/L and 70 g/L by weight of protein.

The protein in the aqueous protein solution may be optionally concentrated and/or separated from small, soluble molecules. Suitable methods for concentrating include, but are not limited to, diafiltration or hydrocyclone. Suitable methods for separation from small, soluble molecules include, but are not limited to, diafiltration.

Salt precipitation may be accomplished using various suitable salts and precipitation pHs. Suitable salts, salt concentrations, polysaccharides, polysaccharide concentrations, and precipitation pHs may be determined by testing various conditions, and identifying the salt and pH and polysaccharide condition which are obtained the most colorless and/or flavorless protein precipitates at the most optimal yield and quality (judged by, for example, by one or more of the following: flavor, odor, color, nitrogen content, Ca content, heavy metal content, emulsification activity, MW distribution, and thermal properties of the protein component obtained). In some embodiments, salt precipitation occurs with calcium dichloride at a concentration of between 5 mM and 1,000 mM. Other examples of suitable salts include, but are not limited to, other alkaline earth metal or divalent salts (e.g., magnesium chloride, sodium chloride, calcium permanganate, and calcium nitrate). Typically, the precipitation pH is opposite the extraction pH (i.e., when the extraction pH is in the basic range, the precipitation pH is most suitable in the acidic range, and vice versa). In some embodiments, the precipitation pH is an acidic pH. In some such embodiments, the acidic pH is less than 7.1, less than 6, less than 5, less than 4, less than 3, less than 2, between 6.9 and 2, between 6 and 3, between 6 and 5, or between 5 and 4. In some such embodiments, the acidic pH is 4. The precipitation pH may be adjusted using a pH adjusting agent. In some embodiments, the pH adjusting agent is a food grade acidic pH adjusting agent. In other embodiments, the pH adjusting agent is a food grade basic pH adjusting agent.

Separating the protein precipitate from non-precipitated components may occur by one or more of the methods disclosed herein.

Washing of the protein precipitate may occur by various methods. In some embodiments, the washing is carried out at the precipitation pH.

The protein precipitate may optionally be suspended. In some embodiments, the suspending is carried out at the extraction pH, for example, in the presence of a chelator to remove calcium ions. If the suspended protein preparation is not transparent it may be clarified by various convenient procedures such as filtration or centrifugation.

The pH of the suspended color-neutral refined protein component may be adjusted to a pH of between 1 and 14, between 2 and 12, between 4 and 10, or between 5 and 7, by the addition of a food grade basic pH adjusting agent, including, for example, sodium hydroxide, or food grade acidic pH adjusting agent, including, for example, hydrochloric acid or phosphoric acid.

The pH of the refined protein component and/or refined protein isolate may be adjusted to a pH of between 1 and 14, between 2 and 12, between 4 and 10, or between 5 and 7, by the addition of a food grade basic pH adjusting agent, including, for example, sodium hydroxide, or food grade acidic pH adjusting agent, including, for example, hydrochloric acid or phosphoric acid.

The refined protein component may be dried. Drying may be performed in a suitable way, including, but not limited to, spray drying, dry mixing, agglomerating, freeze drying, microwave drying, drying with ethanol, evaporation, refractory window dehydration or combinations thereof.

The refined protein component and/or refined protein isolate may be dried. Drying may be performed in a suitable way, including, but not limited to, spray drying, dry mixing, agglomerating, freeze drying, microwave drying, drying with ethanol, evaporation, refractory window dehydration or combinations thereof.

Other optional steps in the methods provided herein are heating steps aimed at removing heat-labile contaminants and/or microbial contaminations, and additional filtering (e.g., carbon filtering) steps aimed at removing additional odor, flavor, and/or color compounds. In some embodiments, such additional filtering is carried out immediately after extracting the protein preparation or after separating the aqueous protein solution from the non-aqueous components.

In some embodiments, the methods provided herein provide a yield of protein of at least 30%, at least 40%, at least 50%, at least 60%, at least 70%, at least 80%, at least 90%, between 30% and 90%%, between 40% and 90%%, between 50% and 90%, or between 60% and 90% by weight.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Preparation of a Deamidated Refined Protein from Pea Flour by Modifying with Protein Glutaminase

This example illustrates a method for preparing a deamidated refined protein useful in the compositions, formulations and methods of the present disclosure.

Materials and methods: Pea Flour (Ingredion, Westchester, Ill., USA) was added to distilled water adjusted to pH 7.0 using 6N NaOH while stirring for approximately 30 to 60 min to a final solid concentration of 20 wt/wt %. The pea flour extract was separated by centrifuging at between 5,000 to 15,000 g for approximately 5 minutes. The supernatant (i.e. the extract) was retained and the pellet was discarded. The supernatant was heated to an inner solution temperature of 45-50° C. Protein glutaminase (Amano PG500, Amano Enzyme USA, Co. Ltd., Elgin, Ill., USA) (10% by weight of protein) was added to the heated supernatant while stirring. The solution was mixed at 45-50° C. for 1 to 5 hours. The deamidation reaction was stopped by heating the solution to have an inner temperature of 75-80° C. for 5 min. The deamidated refined pea protein solution was cooled down to 60° C. and precipitated by adding CaCl₂) and adjusting to a final pH of 4 with 6N HCl. The resulting precipitate was separated by centrifuging between 5,000 to 15,000 g for approximately 10 minutes. The supernatant was discarded, and the precipitate was washed once with water at pH 4.0 using 6N HCl when necessary. The final wash supernatant was discarded, and protein paste collected. The collected deamidated refined protein paste was then used directly in preparing non-dairy analog and beverage formulations as described elsewhere herein.

### Example 2: Feathering in Coffee of a Non-Dairy Analog Made with a Deamidated Refined Protein Component

This example illustrates a study to compare the feathering characteristics in coffee of a non-dairy analog prepared using either unmodified refined pea protein or deamidated refined pea protein.

Materials and methods: Non-dairy analogs were prepared using either unmodified refined pea protein or deamidated refined pea protein. The deamidated pea protein was prepared as provided in Example 1. Both the unmodified refined pea protein or deamidated refined pea protein had a protein content of about 3% by weight within the non-dairy analog. Coffee was heated to between 65-70° C. prior to mixing with the non-dairy analog. The non-dairy analog was between 2-8° C. 80 mL of coffee (pH approximately 5.15) was poured into a beaker and non-dairy analog (2 g) was added. Solutions were stirred clockwise 5 times and an additional 5 times counterclockwise. Images were taken within the first minute and 5 minutes after mixing. To determine if there were differences in non-dairy analog dispersion within the acidic aqueous mixture (coffee), color analysis of the samples was also carried out. To perform the color analysis the coffee samples containing the non-dairy analog were allowed to settle and color analysis was conducted on the supernatant. The color of a food product is determined using a colorimeter or spectrophotometer that measures light reflectance and the L*a*b color space. In this example, the color of the samples was determined using a Datacolor 45S portable spectrophotometer (Datacolor, Lawrenceville, N.J., USA) using illuminant D65 and a visual angle of 10 degrees. A reference tile was used for calibration, and the results were expressed using the CIELAB system (determining L*—lightness, a* (green/red), and b* (blue/yellow)).

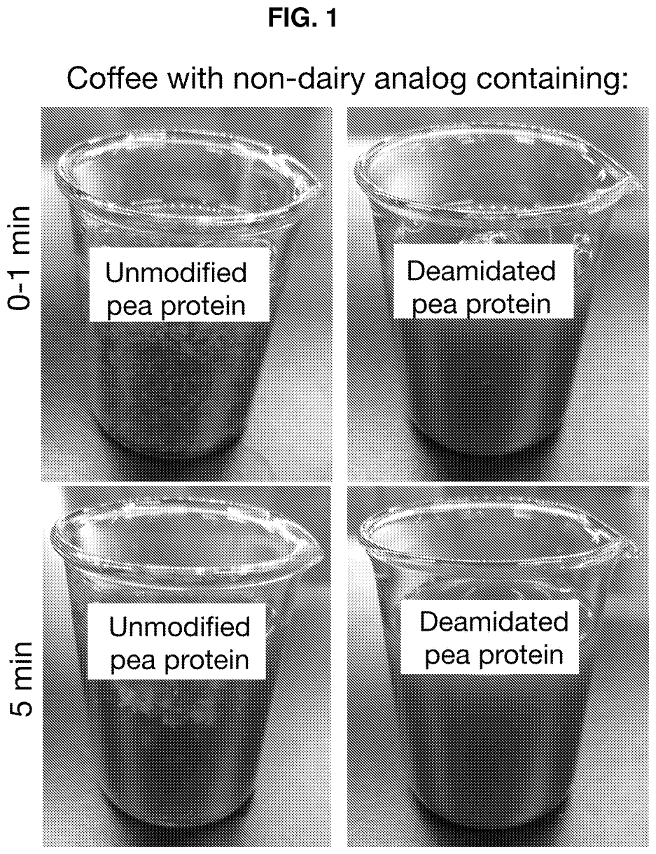

Results: FIG. 1 shows images taken between 0-1 min. (top) and 5 min. (bottom) after addition of non-dairy analog to coffee. The sample containing the non-dairy analog made with unmodified pea protein is shown at left, and the sample containing the non-dairy analog made using deamidated refined pea protein is shown at right. Feathering occurred instantly with the non-dairy analog containing unmodified refined pea protein. This feathering settled to the bottom of the coffee, which left a color profile of the existing supernatant closer to that of traditional coffee. Alternatively, the non-dairy analog containing deamidated refined pea protein did not feather within coffee. This result was further confirmed with the color of the supernatant mixture being whiter and “creamier” looking in comparison to traditional coffee.

The results of the color analysis are provided below in Table 1.

TABLE 1
Data color analysis coffee and/or non-dairy analogs

| Sample | L* | a* | b* |
|---|---|---|---|
| Coffee | 3.63 | −0.09 | −1.20 |
| Coffee + Unmodified non-dairy analog | 4.45 | −0.11 | −1 |
| Coffee + Deamidated non-dairy analog | 14.19 | 3.3 | 7.63 |

### Example 3: Evaluating the Ranges of Protein Glutaminase Refined Protein Needed to Decrease Feathering of Non-Dairy Analog in Acidic Aqueous Composition (e.g., Coffee)

This example illustrates a study showing that the amount of feathering in coffee due to mixing with a non-dairy analog decreases with an increase in the percentage of deamidated refined protein used in the non-dairy analog.

Materials and methods: Both unmodified and deamidated non-dairy analogs were prepared using either unmodified refined pea protein or deamidated refined pea protein. The deamidated refined pea protein used in this example was prepared as provided in Example 1. Both the unmodified refined pea protein non-dairy analog or deamidated refined pea protein non-dairy analog had a protein content of about 3% by weight. Both non-dairy analogs were mixed at different ratios from 20-100% before proceeding with coffee stability analysis. Coffee was heated to between 65-70° C. prior to mixing with the non-dairy milks. The non-dairy milk was between 2-8° C. Coffee (10 mL) was poured into a beaker and initial pH checked to be about 5.15. Non-dairy analog (0.25 g) was added followed by stirring clockwise 5 times and an additional 5 times counterclockwise. Samples were allowed to sit 10 minutes before transferring into graduated centrifuge tubes. Transferred samples were allowed to sit an additional 5 minutes before centrifugation at 164 g for 5 minutes. Feathered wet solid volumes were then recorded to the nearest 0.1 mL. These wet solids are also referred to herein as precipitate.

Results: As shown by the results summarized in Table 2, the amount of feathering wet solids (mL) in coffee following the addition of a non-dairy analog decreased as the percentage of deamidated non-dairy analog increased.

TABLE 2
Feathering decrease correlation with deamidated non-dairy analog

| Sample Label | Unmodified | Deamidated | Feathering |
|---|---|---|---|
| (Unmodified %- | non-dairy | non-dairy | wet |
| Deamidated %) | analog (%) | analog (%) | solids (mL) |
| 100%-0% | 100 | 0 | 0.5 |
| 80%-20% | 80 | 20 | 0.4 |
| 60%-40% | 60 | 40 | 0.3 |
| 40%-60% | 40 | 60 | 0.3 |
| 20%-80% | 20 | 80 | 0.1 |
| 0%-100% | 0 | 100 | 0 |

### Example 4: Preparation of a Non-Dairy Analog

This example illustrates compositions and methods for preparing non-dairy analog products using an unmodified or a deamidated refined pea protein component of the present disclosure.

An exemplary non-dairy analog can be formulated and prepared based on the composition of ingredients shown in Table 3.

TABLE 3
Composition of Non-Dairy Analog

| Ingredient | Supplier | % by weight |
|---|---|---|
| Water, filtered | | 83-94 |
| Refined protein component | Deamidated and/or unmodified | 0.5-5 |
| (including any ratio of unmodified to | refined pea proteins e.g., as | |
| deamidated pea protein, e.g., 100:0, | prepared in Example 1 | |
| 90:10, 80:20, 70:30, 60:40, 50:50, | | |
| 40:60, 30:70, 20:80, 10:90, 0:100.) | | |
| Organic Expeller Pressed | Spectrum (Boulder, CO) | 2-10 |
| Sunflower Oil | | |
| Phosphate salts | ICL (Tel Aviv, Israel) | 0.3-1 |
| Flavoring agents (e.g., natural milk | | 0-1 |
| type flavor and natural butter type | | |
| flavor) | | |
| Potassium Hydroxide, 45% | | 0.02-1.00 |
| Gellan gum | CP Kelco (Atlanta, GA) | 0.01-0.05 |
| Cane Sugar | | 0-2 |

Materials and methods: Phosphate salts, dry flavor, sugar, and gums were combined in a dry blend. Water added, and solution mixed under high shear (5,000 rpm, 5-10 min) until dissolved, the deamidated refined protein component was then added, and the mixture was mixed for approximately 5 minutes at ˜5,000 rpm to obtain a liquid blend. Oil added to the mixture and high shear mixed an additional 5-10 minutes at 5,000 rpm. The pH was recorded, and potassium hydroxide was added to bring the pH to 8.3 (+/−0.05 @ 40° F.). The blend was heated to 90 C and subjected to homogenization (two stage, 2500 psi). The resulting product was collected and cooled.

Table 3 provides ranges of values that are contemplated for use in preparing a non-dairy analog of the present disclosure. For example, certain exemplary embodiments may use between 0-30% by weight of unmodified refined protein with between 100-70% by weight of the deamidated refined protein. In addition, slight variations in the formulation of the non-dairy analog of the present example are also contemplated. For example, different flavoring agents (e.g., natural sweetness enhancer flavor and natural chocolate type flavor, or natural vanilla flavor and vanilla extract) may be used to produce flavored non-dairy analogs (e.g., chocolate non-dairy analog or vanilla non-dairy-analog, respectively). Additionally, different phosphate salts (e.g., tricalcium phosphate, potassium phosphate, dipotassium phosphate, sodium phosphate, and disodium phosphate) may be used to produce specific non-dairy analogs (e.g., milk, barista style milk, and creamer).

### Example 5: Deamidation of Protein Component During Non-Dairy Analog Production

This example illustrates a method in which deamidation of the refined protein component occurs during the process for preparing a non-dairy analog containing the refined protein.

Table 4 provides ranges of ingredients contemplated for use in the deamidated non-dairy analog preparation method of this example. It is contemplated that different flavoring agents (e.g., natural sweetness enhancer flavor and natural chocolate type flavor, or natural vanilla flavor and vanilla extract) may be used to produce flavored non-dairy analogs (e.g., chocolate non-dairy analog or vanilla non-dairy-analog, respectively). Additionally, different phosphate salts (e.g., tricalcium phosphate, potassium phosphate, dipotassium phosphate, sodium phosphate, and disodium phosphate) may be used to produce specific non-dairy analogs (e.g., milk, barista style milk, and creamer).

TABLE 4
Composition of Non-Dairy Analog

| Ingredient | Supplier | % by weight |
|---|---|---|
| Water, filtered | | 83-94 |
| Refined protein component | Spray-dried unmodified | 0.5-5 |
| | refined pea protein | |
| | component prepared as in | |
| | Example 1 | |
| Organic Expeller Pressed | Spectrum (Boulder, CO) | 2-10 |
| Sunflower Oil | | |
| Phosphate salts | ICL (Tel Aviv, Israel) | 0.3-1 |
| Flavoring agents (e.g., natural | | 0-1 |
| milk type flavor and natural | | |
| butter type flavor) | | |
| Potassium Hydroxide, 45% | | 0.02-0.15 |
| Kelcogel HA-B Gellan | CP Kelco (Atlanta, GA) | 0.01-0.05 |
| Organic Cane Sugar | | 0-2 |
| Protein Glutaminase | Amano PG500 | 0-0.3 |

Materials and Methods:

A. Non-Dairy Analog Preparation

Refined pea protein isolate was prepared as described in Example 1 (i.e., without the glutaminase treatment) and spray-dried. The spray-dried unmodified refined pea protein (0.5-5 wt %) and phosphate salts (0.3-1 wt %) were mixed in water. The mixture was pH adjusted to pH 8.0-8.5 with 45% KOH. The solution was continually mixed and heated to 45-50° C. Protein glutaminase (0.1-0.3 wt %) was added and the solution was mixed at 45-50° C. for 45-180 min. After the desired incubation time, flavors (0-1 wt %), sugar (0-2 wt %), gums (0.01-0.05 wt %), and oil (2-10 wt %) were added to the mixing solution. The blend was then heated to 90° C. and subjected to homogenization (two stage, 2500 psi). The resulting product was collected and cooled.

B. Feathering Analysis

Coffee was heated to between 65-70° C. prior to mixing with the non-dairy analog samples. The non-dairy analogs were between 2-8° C. Coffee (50 mL) was poured into a beaker and initial pH checked to be about 5.15. Each non-dairy analog (0.5-1.5 mL) was added followed by stirring clockwise 5 times and an additional 5 times counterclockwise. The samples were allowed to sit 10 minutes before transferring into graduated centrifuge tubes. Transferred samples were allowed to sit an additional 5 minutes before centrifugation at 160 g for 5 minutes. Feathered wet solid volumes were then recorded to the nearest 0.1 mL.

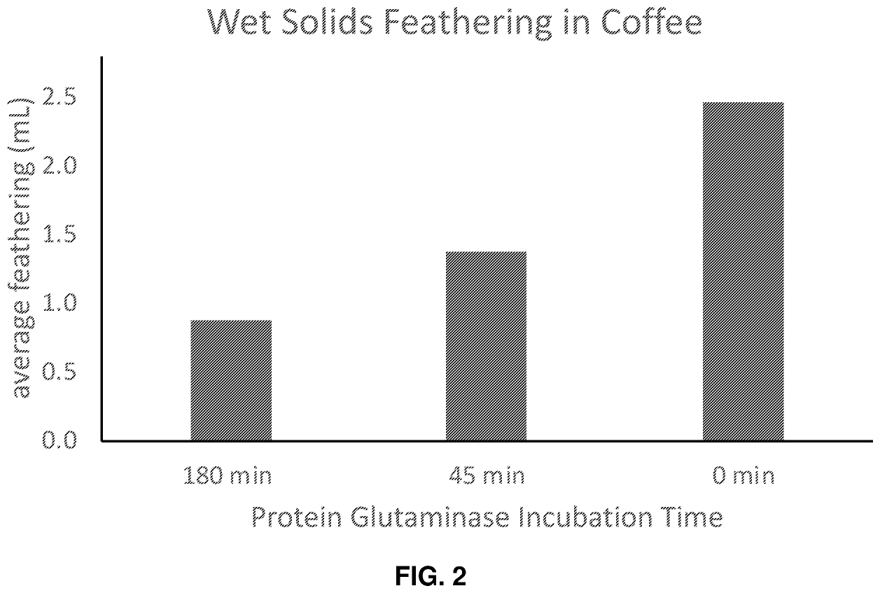

Results: As shown in FIG. 2, there was a significant decrease in feathering exhibited in coffee by the non-dairy analog that was prepared with the longest incubation in glutaminase, which results in the greatest deamidation of the refined pea protein component. The amount of wet solids due to feathering decreased by approximately 45% (from 2.5 mL to ˜1.3 mL) after 45 minutes of glutaminase incubation, and approximately 70% (from 2.5 mL to ˜0.8 mL) after 180 minutes of glutaminase incubation.

Notwithstanding the appended claims, the disclosure set forth herein is also defined by the following clauses, which may be beneficial alone or in combination, with one or more other causes or embodiments. Without limiting the foregoing description, certain non-limiting clauses of the disclosure numbered as below are provided, wherein each of the individually numbered clauses may be used or combined with any of the preceding or following clauses. Thus, this is intended to provide support for all such combinations and is not necessarily limited to specific combinations explicitly provided below:

A1. A non-dairy analog, the non-dairy analog comprising: (a) a refined protein component in which at least a portion of the refined protein component is a deamidated refined protein component; (b) at least one lipid in which the at least one lipid is from a non-animal natural source; (c) at least one emulsifier; (c) water; and (d) a pH of between 6.5 and 10.

A2. A non-dairy analog, the non-dairy analog comprising: (a) at least 0.5% by weight of a refined protein component in which at least 30% by weight of the refined protein component is a deamidated refined protein component; (b) between 1% by weight and 10% by weight of at least one lipid in which the at least one lipid is from a non-animal natural source; (c) between 0.01% by weight and 10% by weight of at least one emulsifier; (d) water; and (e) a pH of between 6.5 and 10.

A3. The non-dairy analog of clauses A1 or A2, wherein the non-dairy analog exhibits less than 5%, 3%, 1% or 0.5% by volume precipitation when added to the aqueous composition after at least partially mixing of the non-dairy analog with the aqueous composition, wherein the aqueous composition has a temperature that is between 30° C. and 95° C. and an aqueous composition pH that is less than 7, before the non-dairy analog is combined with the aqueous composition or after the non-dairy analog is combined with the aqueous composition.

A4. The non-dairy analog of clauses A1 or A2, wherein the non-dairy analog exhibits less than 5%, 3%, 1% or 0.5% by volume precipitation when added to the aqueous composition after at least partially mixing of the non-dairy analog with the aqueous composition, wherein the aqueous composition has a temperature that is between 30° C. and 95° C. and an aqueous composition pH that is less than 7, before the non-dairy analog is combined with the aqueous composition or after the non-dairy analog is combined with the aqueous composition.

A5. The non-dairy analog of one or more of clauses A1 to A4, wherein non-dairy analog is capable of being solubilized and/or dispersed, or substantially solubilized and/or substantially dispersed, in the aqueous composition 15 minutes, 10 minutes, or 5 minutes after at least partially mixing of the non-dairy analog composition with the aqueous composition.

A6. The non-dairy analog of one or more of clauses A1 to A5, wherein the non-dairy analog exhibits less than 5%, 3%, 1% or 0.5% by volume precipitation when added to the aqueous composition after at least partially mixing of the non-dairy analog with the aqueous composition 15 minutes, 10 minutes, or 5 minutes after at least partially mixing of the non-dairy analog composition with the aqueous composition.

A7. The non-dairy analog of one or more of clauses A1 to A6, wherein the non-dairy analog is capable of not visibly precipitating when added to the aqueous composition for 15 minutes, 10 minutes, or 5 minutes after at least partially mixing of the non-dairy analog composition with the aqueous composition.

A8. The non-dairy analog of one or more of clauses A1 to A7, wherein the non-dairy analog exhibits insubstantial precipitation when added to the aqueous composition for 15 minutes, 10 minutes, or 5 minutes after at least partially mixing of the non-dairy analog composition with the aqueous composition.

A9. The non-dairy analog of one or more of clauses A1 to A8, wherein the non-dairy analog exhibits less than 5%, 3%, 1% or 0.5% by volume precipitation when added to the aqueous composition after at least partially mixing of the non-dairy analog with the aqueous composition.

A10. The non-dairy analog of one or more of clauses A1 to A9, wherein the non-dairy analog is capable of not visibly feathering when added to the aqueous composition for 15 minutes, 10 minutes, or 5 minutes after at least partially mixing of the non-dairy analog with the aqueous composition.

A11. The non-dairy analog of one or more of clauses A1 to A10, wherein the deamidated refined protein component is at least 30%, 40%, 50%, 60%, 70%, 80%, 90% or 100% by weight of the total weight of the refined protein component.

A12. The non-dairy analog of one or more of clauses A1 to A10, wherein the deamidated refined protein component is between 30% to 100%, 40% to 100%, 40% to 60%, 40% to 70% or 30% to 50% by weight of the total weight of the refined protein component.

A13. The non-dairy analog of one or more of clauses A1 to A12, wherein the refined protein component is sourced from a legume.

A14. The non-dairy analog of one or more of clauses A1 to A12, wherein the refined protein component is sourced from a pea plant or a pea protein.

A15. The non-dairy analog of one or more of clauses A1 to A12, wherein the refined protein component is a refined pea protein component in which at least a portion of the refined pea protein component is the deamidated refined pea protein component.

A16. The non-dairy analog of one or more of clauses A1 to A15, wherein the pH of the non-dairy analog is between 6.5 and 7.9, 7 and 7.9 or 7.5 and 8.3.

A17. The non-dairy analog of one or more of clauses A1 to A16, wherein the pH of the non-dairy analog is at least 7.3, 7.4, 7.5, 7.6, 7.7, 7.8, 7.9, 8.0, 8.1, 8.2, or 8.3.

A18. The non-dairy analog of one or more of clauses A1 to A17, wherein non-dairy analog has at least 0.2%, 0.5%, 1%, 2%, 3%, 4%, 5%, 8% or 10% by weight of the refined protein component.

A19. The non-dairy analog of one or more of clauses A1 to A18, wherein the refined protein component of the non-dairy analog is between 0.2% to 5%, 0.5% to 5%, 0.5% to 4%, 0.5% to 3%, or 1% to 4% by weight.

B1. A method of using a non-dairy analog component comprising; 1) combining a non-dairy analog with an aqueous composition where the aqueous composition is between 30° C. and 95° C. and has a pH of less than 7 prior to being combined with the non-dairy analog; and 2) at least partially mixing the non-dairy analog with the aqueous composition in order to at least partially distribute the non-dairy analog with the aqueous composition, wherein the non-dairy analog comprises: (a) a refined protein component in which at least a portion of the refined protein component is a deamidated refined protein component; (b) at least one lipid in which the at least one lipid is from a non-animal natural source; (c) at least one emulsifier; (d) water; and (e) a pH of between 6.5 and 10; wherein the non-dairy analog is substantially soluble when combined with the aqueous composition.

B2. A method of using a non-dairy analog comprising; 1) combining a non-dairy analog with an aqueous composition where the aqueous composition is between 30° C. and 95° C. and has a pH of less than 7 prior to being combined with the non-dairy analog; and 2) at least partially mixing the non-dairy analog with the aqueous composition in order to at least partially distribute the non-dairy analog with the aqueous composition, wherein the non-dairy analog comprises: (a) at least 0.5% by weight by weight of a refined protein component in which at least 30% by weight of the refined protein component is a deamidated refined protein component; (b) between 1% by weight and 10% by weight of at least one lipid in which the at least one lipid is from a non-animal natural source; (c) between 0.01% by weight and 10% by weight of at least one emulsifier; (d) water; and (e) a pH of between 6.5 and 10; wherein the non-dairy analog is soluble, or substantially soluble, when combined with the aqueous composition.

B3. The method of clause B2, wherein the aqueous composition is a coffee.

B4. The method of clause B2, wherein the aqueous composition is an infusion, for example tea.

B5. The method of clause B2, wherein the aqueous composition is a hot chocolate.

B6. The method of one or more of clauses B2 to B5, wherein the non-dairy analog exhibits less than 5%, 3%, 1% or 0.5% by volume precipitation when added to the aqueous composition after at least partially mixing of the non-dairy analog with the aqueous composition.

B7. The method of one or more of clauses B2 to B5, wherein the non-dairy analog exhibits less than 1% or 0.5% by volume precipitation when added to the aqueous composition after at least partially mixing of the non-dairy analog with the aqueous composition.

B8. The method of one or more of clauses B2 to B7, wherein non-dairy analog is soluble, or substantially soluble, in the aqueous composition 15 minutes, 10 minutes, or 5 minutes after at least partially mixing of the non-dairy analog with the aqueous composition.

B9. The method of one or more of clauses B2 to B7, wherein the non-dairy analog exhibits less than 5%, 3%, 1% or 0.5% by volume precipitation when added to the aqueous composition.

B10. The method of one or more of clauses B2 to B7, wherein the non-dairy analog exhibits less than 1% or 0.5% by volume precipitation when added to the aqueous composition.

B11. The method of clauses B9 or B10, wherein the refined protein component in the non-dairy analog is soluble, or substantially soluble, in the aqueous composition 15 minutes, 10 minutes, or 5 minutes after at least partially mixing of the non-dairy analog with the aqueous composition.

B12. The method of one or more of clauses B2 to B11, wherein the non-dairy analog is stable in the aqueous composition after at least partially mixing of the non-dairy analog with the aqueous composition.

B13. The method of clause B12, wherein the non-dairy analog exhibits less than 5%, 3%, 1% or 0.5% by volume precipitation when added to the aqueous composition after at least partially mixing of the non-dairy analog with the aqueous composition.

B14. The method of clauses B12 or B13, wherein the non-dairy analog is stable in the aqueous composition 15 minutes, 10 minutes, or 5 minutes after at least partially mixing of the non-dairy analog with the aqueous composition.

B15. The method of one or more of clauses B2 to B14, wherein the refined protein component in the non-dairy analog is stable in the aqueous composition after at least partially mixing of the non-dairy analog with the aqueous composition.

B16. The method of clause B15, wherein the refined protein is at least substantially stable in the aqueous composition after at least partially mixing of the non-dairy analog with the aqueous composition.

B17. The method of clauses B15 or B16, wherein the refined protein component in the non-dairy analog is stable in the aqueous composition 15 minutes, 10 minutes, or 5 minutes after at least partially mixing of the non-dairy analog with the aqueous composition.

B18. The method of one or more of clauses B2 to B17, wherein the non-dairy analog does not visibly precipitate when added to the aqueous composition.

B19. The method of clause B18, wherein the non-dairy analog does not visibly precipitate when added to the aqueous composition for 15 minutes, 10 minutes, or 5 minutes after at least partially mixing of the non-dairy analog with the aqueous composition.

B20. The method of one or more of clauses B2 to B19, wherein the refined protein component in the non-dairy analog does not visibly precipitate when added to the aqueous composition.

B21. The method of clause B20, wherein the refined protein component in the non-dairy analog does not visibly precipitate when added to the aqueous composition 15 minutes, 10 minutes, or 5 minutes after at least partially mixing of the non-dairy analog with the aqueous composition.

B22. The method of one or more of clauses B2 to B21, wherein the non-dairy analog exhibits insubstantial precipitation when added to the aqueous composition after at least partially mixing of the non-dairy analog with the aqueous composition.

B23. The method of example B22, wherein the non-dairy analog exhibits insubstantial precipitation when added to the aqueous composition for 15 minutes, 10 minutes, or 5 minutes after at least partially mixing of the non-dairy analog with the aqueous composition.

B24. The method of one or more of clauses B2 to B23, wherein the non-dairy analog exhibits less than 5%, 3%, 1% or 0.5% by volume precipitation when added to the aqueous composition after at least partially mixing of the non-dairy analog with the aqueous composition.

B25. The method of one or more of clauses B2 to B24, wherein the non-dairy analog does not visibly feather when added to the aqueous composition after at least partially mixing of the non-dairy analog with the aqueous composition.

B26. The method of clause B25, wherein the non-dairy analog does not visibly feather when added to the aqueous composition for 15 minutes, 10 minutes, or 5 minutes after at least partially mixing of the non-dairy analog with the aqueous composition.

B27. The method of one or more of clauses B2 to B24, wherein the non-dairy analog exhibits insubstantial visibly feathering when added to the aqueous composition after at least partially mixing of the non-dairy analog with the aqueous composition.

B28. The method of clause B27, wherein the non-dairy analog does not visibly feather when added to the aqueous composition for 15 minutes, 10 minutes, or 5 minutes after at least partially mixing of the non-dairy analog with the aqueous composition.

B29. The method of one or more of clauses B2 to B24, wherein the non-dairy analog exhibits less than 5%, 3%, 1% or 0.5% by volume feathering when added to the aqueous composition.

B28. The method of clause B29, wherein the non-dairy analog exhibits less than 5%, 3%, 1% or 0.5% by volume feathering when added to the aqueous composition for 15 minutes, 10 minutes, or 5 minutes after at least partially mixing of the non-dairy analog with the aqueous composition.

B29. The method of one or more of clauses B2 to B24, wherein the refined protein component in the non-dairy analog exhibits less than 5%, 3%, 1% or 0.5% by volume feathering when added to the aqueous composition after at least partially mixing of the non-dairy analog with the aqueous composition.

B30. The method of clause B29, wherein the refined protein component in the non-dairy analog non-dairy analog exhibits less than 5%, 3%, 1% or 0.5% by volume feathering when added to the aqueous composition for 15 minutes, 10 minutes, or 5 minutes after at least partially mixing of the non-dairy analog with the aqueous composition.

B31. The method of one or more of clauses B1 to B30, wherein the refined protein component is at least 0.5%, 1%, 4%, 4.5%, 5%, 10%, 15% or 20% by weight of the total weight of the non-dairy analog.

B32. The method of one or more of clauses B1 to B30, wherein the refined protein component is between 0.5% and 20%, 4% and 10%, 4% and 5%, 5% and 15% or 10% and 16% by weight of the total weight of the non-dairy analog.

B33. The method of one or more of clauses B1 to B32, wherein the deamidated refined protein component is at least 20%, 30%, 40%, 50%, 60%, 70%, 80%, 90% or 100% by weight of the total weight of the refined protein component.

B34. The method of one or more of clauses B1 to B32, wherein the deamidated refined protein component is between 40% to 60%, 20% to 70%, 30% to 80%, 35% to 90%, 20% to 60% or 40% to 100% by weight of the total weight of the refined protein component.

B35. The method of one or more of clauses B1 to B34, wherein the refined protein component is sourced from a plant.

B36. The method of one or more of clauses B1 to B35, wherein the refined protein component is sourced from a legume.

B37. The method of one or more of clauses B1 to B36, wherein the refined protein component is sourced from a pea plant or a pea protein.

B38. The method of one or more of clauses B1 to B37, wherein the refined protein component is a refined pea protein component in which at least a portion of the refined pea protein component is a deamidated refined pea protein component.

B39. The method of one or more of clauses B1 to B38, wherein non-dairy analog has a pH between 6.5 and 7.9, 7 and 7.9 or 7.5 and 8.3.

B40. The method of one or more of clauses B1 to B39, wherein non-dairy analog has a pH at least 7.3, 7.4, 7.5, 7.6, 7.7, 7.8, 7.9, 8.0, 8.1, 8.2, or 8.3.

B41. The method of one or more of clauses B1 to B40, wherein the refined protein component is a color-neutral refined protein component.

B42. The method of one or more of clauses B1 to B41, wherein the refined protein component is not a color-neutral refined protein component.

C1. A non-dairy analog comprising: (a) a refined protein component in which at least a portion of the refined protein component is a deamidated refined protein component; (b) at least one lipid from a non-animal natural source; (c) at least one emulsifier; (d) water; and (e) a pH of between 4.0 and 10, optionally, a pH of between 6.5 and 10.

C2. The non-dairy analog of clause 1, wherein: (a) at least 0.5% by weight of a refined protein component in which at least 10% by weight of the refined protein component is a deamidated refined protein component; (b) between 1% by weight and 10% by weight of at least one lipid from a non-animal natural source; and/or (c) between 0.01% by weight and 10% by weight of at least one emulsifier.

C3. The non-dairy analog of any one of clauses C1 or C2, wherein the refined protein component comprises is at least 0.2%, 0.5%, 1%, 2%, 3%, 5%, 8% or 10% by weight of the non-dairy analog; optionally, between 0.5% to 5%, 3% to 4%, 0.2% to 10%, 1% to 10%, 1% to 5%, 3% to 8% or 2% to 4% by weight of the non-dairy analog.

C4. The non-dairy analog of any one of clauses C1 to C3, wherein the deamidated refined protein component is at least 10%, 20%, 30%, 40%, 50%, 60%, 70%, 80%, 90% or 100% by weight of the total weight of the refined protein component; optionally, wherein the deamidated refined protein component is between 10% to 100%, 20% to 90%, 30% to 80%, 40% to 70%, or 30% to 50% by weight of the total weight of the refined protein component.

C5. The non-dairy analog of any one of clauses C1 to C4, wherein the deamidated refined protein component is deamidated by glutaminase treatment; optionally, wherein the glutaminase treatment is carried out using a protein-glutaminase; optionally, wherein the protein-glutaminase is isolated or derived from C. proteolyticum.

C6. The non-dairy analog of any one of clauses C1 to C5, wherein the glutaminase treatment comprises at least 0.1 wt %, at least 0.2 wt %, at least 0.3 wt %, at least 0.4 wt %, at least 0.5 wt %, at least 0.75 wt %, at least 1.0 wt %, at least 2.5 wt %, at least 5.0 wt %, at least 7.5 wt %, at least 10 wt %, or at least 15 wt % glutaminase relative to refined protein; optionally, between about 0.1 wt % and 15 wt %, between about 0.1 wt % and 10 wt %, between about 0.1 wt % and 5.0 wt %, between about 0.1 wt % and 2.5 wt %, or between about 0.1 wt % and 1.0 wt % glutaminase relative to refined protein.

C7. The non-dairy analog of any one of clauses C1 to C6, wherein the refined protein component is sourced from a plant; optionally, sourced from a legume.

C8. The non-dairy analog of any one of clauses C1 to C7, wherein the refined protein component is sourced from a pea plant or a pea protein.

C9. The non-dairy analog of any one of clauses C1 to C8, wherein the pH of the non-dairy analog is at least 4.0, 6.5, 7.3, 7.4, 7.5, 7.6, 7.7, 7.8, 7.9, 8.0, 8.1, 8.2, or 8.3; optionally, wherein the pH is between 4.0 and 8.3, 4.0 and 7.9, 6.5 and 7.9, 7 and 7.9, or 7.5 and 8.3. wherein the pH of the non-dairy analog.

C10. The non-dairy analog of any one of clauses C1 to C9, wherein after at least partial mixing with an aqueous solution, the non-dairy analog: (a) exhibits no visible feathering for at least 5 minutes, 10 minutes, or 15 minutes after mixing; (b) exhibits no visible precipitation for at least 5 minutes, 10 minutes, or 15 minutes after mixing; (c) exhibits insubstantial precipitation for at least 5 minutes, 10 minutes, or 15 minutes after mixing; (d) exhibits less than 5%, 3%, 1% or 0.5% by weight precipitation; optionally, wherein the by weight precipitation is exhibited for at least 5 minutes, 10 minutes, or 15 minutes after mixing; (e) exhibits less than 5%, 3%, 1% or 0.5% by volume precipitation; optionally, wherein the by volume precipitation is exhibited for at least 5 minutes, 10 minutes, or 15 minutes after mixing; and/or (f) is solubilized, or substantially solubilized, for at least 5 minutes, 10 minutes, or 15 minutes after mixing.

C11. The non-dairy analog of any one of clauses C1 to C10, wherein the aqueous composition has a temperature between 30° C. and 95° C.

C12. The non-dairy analog of any one of clauses C1 to C11, wherein the aqueous composition has a pH of less than 7, before the non-dairy analog is mixed with it; optionally wherein the aqueous composition has a pH less than 7 after the non-dairy analog is mixed with it.

C13. The non-dairy analog of any one of clauses C1 to C12, wherein the total solubility and/or dispersibility of the refined protein component of the non-dairy analog when combined with an aqueous composition is increased at least 10%, 20%, 50%, 60%, or 70% relative to the non-dairy analog without the deamidated refined protein component.

C14. The non-dairy analog of clause C13, wherein: (a) the aqueous composition pH is between 3 to 10, before the non-dairy analog is combined with the aqueous composition; (b) the aqueous composition pH is less than 7, before the non-dairy analog is combined with the aqueous composition; (c) wherein the aqueous composition pH is between 3 and 6, before the non-dairy analog is combined with the aqueous composition; (d) wherein the aqueous composition has a temperature of between 30 degrees Celsius and 95 degrees Celsius the aqueous composition pH is between 3 and 10, before the non-dairy analog is combined with the aqueous composition; (e) wherein the aqueous composition has a temperature of between 30 degrees Celsius and 95 degrees Celsius and the aqueous composition pH is less than 7, before the non-dairy analog is combined with the aqueous composition; or (f) wherein the aqueous composition has a temperature of between 30 degrees Celsius and 95 degrees Celsius and the aqueous composition pH of between 3 and 6, before the non-dairy analog is combined with the aqueous composition.

D1. A method for producing a non-dairy analog, the method comprising: (a) blending with water to generate a mixture (i) at least one lipid from a non-animal natural source, and (ii) at least one refined protein component from a non-animal natural source, wherein at least a portion of the refined protein component comprises a deamidated refined protein; and (b) emulsifying at least a portion of the mixture to provide a non-dairy analog.

D2. The method of clause D1, wherein the deamidated refined protein component is deamidated by glutaminase treatment; optionally, wherein the glutaminase treatment is carried out using a protein-glutaminase; optionally, wherein the protein-glutaminase is isolated or derived from C. proteolyticum.

D3. The method of clause D2, wherein the glutaminase treatment comprises at least 0.1 wt %, at least 0.2 wt %, at least 0.3 wt %, at least 0.4 wt %, 0.5 wt %, at least 0.75 wt %, at least 1.0 wt %, at least 2.5 wt %, at least 5.0 wt %, at least 7.5 wt %, at least 10 wt %, or at least 15 wt % glutaminase relative to refined protein; optionally, between about 0.1 wt % and 15 wt %, between about 0.1 wt % and 10 wt %, between about 0.1 wt % and 5.0 wt %, between about 0.1 wt % and 2.5 wt %, or between about 0.1 wt % and 1.0 wt % glutaminase relative to refined protein.

D4. The method of any one of clauses D1 to D3, wherein the refined protein component is sourced from a plant; optionally, sourced from a legume.

D5. The method of any one of clauses D1 to D4, wherein the refined protein component is sourced from a pea plant or a pea protein.

D6. The method of any one of clauses D1 to D5, wherein: (a) at least 2% by weight of a refined protein component in which at least 10% by weight of the refined protein component is a deamidated refined protein component; (b) between 1% by weight and 10% by weight of at least one lipid from a non-animal natural source; and/or (c) between 0.01% by weight and 5% by weight of at least one emulsifier.

D7. The method of any one of clauses D1 to D6, wherein the refined protein component comprises is at least 0.5%, 1%, 2%, 3%, 5%, 8% or 10% by weight of the non-dairy analog; optionally, between 2% to 5%, 3% to 4%, 0.5% to 10%, 1% to 5%, 3% to 8% or 2% to 4% by weight of the non-dairy analog.

D8. The method of any one of clauses D1 to D7, wherein the deamidated refined protein component is at least 10%, 20%, 30%, 40%, 50%, 60%, 70%, 80%, 90% or 100% by weight of the total weight of the refined protein component; optionally, wherein the deamidated refined protein component is between 10% to 100%, 20% to 100%, 30% to 80%, 40% to 70% or 30% to 50% by weight of the total weight of the refined protein component.

E1. A beverage formulation comprising: (a) a refined protein component in which at least a portion of the refined protein component is a deamidated refined protein component; (b) water or carbonated water; and (c) a pH of between 6 and 9.

E2. The beverage formulation of clause E1, wherein the deamidated refined protein component is at least 10%, 20%, 30%, 40%, 50%, 60%, 70%, 80%, 90% or 100% by weight of the total weight of the refined protein component; optionally, wherein the deamidated refined protein component is between 10% to 100%, 20% to 90%, 30% to 80%, 40% to 70% or 30% to 50% by weight of the total weight of the refined protein component.

E3. The beverage formulation of any one of clauses E1 to E2, wherein the refined protein component comprises is at least 0.2%, 0.5%, 1%, 2%, 3%, 5%, 8% or 10% by weight of the beverage formulation; optionally, between 0.2% to 10%, 0.5% to 10%, 1% to 5%, 2% to 5%, 3% to 4%, 3% to 8%, or 2% to 4% by weight of the beverage formulation.

E4. The beverage formulation of any one of clauses E1 to E3, wherein the beverage: (a) exhibits less than 5%, 3%, 1% or 0.5% by weight precipitation of the refined protein component; and/or (b) exhibits less than 5%, 3%, 1% or 0.5% by volume precipitation of the refined protein component.

E5. The beverage formulation of any one of clauses E1 to E4, wherein the refined protein component is sourced from a plant; optionally, sourced from a legume.

E6. The beverage formulation of any one of clauses E1 to E5, wherein the refined protein component is sourced from a pea plant or a pea protein.

E7. The beverage formulation of any one of clauses E1 to E6, wherein the beverage formulation further comprises: (a) sugar and/or a carbohydrate; (b) at least one vitamin or mineral; (c) at least one lipid from a non-animal natural source; and/or (d) at least one emulsifier, and/or a hydrocolloid or gum.

E8. The beverage formulation of any one of clause E1 to E7, wherein the beverage formulation is selected from: a protein drink, a vitamin drink, a fruit juice drink, or an iced tea drink.

F1. A method for preparing a non-dairy analog, the method comprising: (a) generating an aqueous mixture at pH 7.5-8.5 and 35-60° C. comprising: an 0.5-5.0 wt % unmodified refined protein component from a non-animal natural source, and 0.3-1.0 wt % buffering salts; (b) adding to the mixture 0.1-1.0 wt % of a glutaminase and mixing at 35-60° C. for at least 1 hour, whereby at least a portion of the unmodified refined protein component is deamidated; (c) after mixing of step (b) adding to the mixture 2-10 wt % oil, 0.01-0.05 wt % gums, 0-1 wt % emulsifier, 0-1 wt % flavors and 0-2 wt % sugar; and (d) heating the mixture to 90° C. and subjecting to homogenization.

F2. The method of clause F1, wherein the refined protein component is sourced from a plant; optionally, sourced from a legume.

F3. The method of any one of clauses F1 to F2, wherein the refined protein component is sourced from a pea plant or a pea protein.

F4. The method of any one of clauses F1 to F3, wherein the buffering salts comprise chloride salts and/or phosphate salts; optionally, wherein the buffering salts are phosphate salts.

F5. The method of any one of clauses F1 to F4, wherein the glutaminase is a protein glutaminase; optionally, wherein the glutaminase comprises a protein-glutaminase isolated or derived from C. proteolyticum.

F6. The method of any one of clauses F1 to F5, wherein steps (a) and (b) are carried out at a temperature of 40-55° C.; optionally, wherein steps (a) and (b) are carried out at a temperature of 45-50° C.

F7. The method of any one of clauses F1 to F6, wherein the step (b) mixing time is from 1 to 12 hours, from 1 to 6 hours, or from 1 to 3 hours; optionally, the mixing time is from 1 to 3 hours.

F8. The method of any one of clauses F1 to F7, wherein the portion of the unmodified refined protein component that is deamidated is at least 10%, at least 20%, at least 30%, at least 40%, at least 50%, at least 60%, at least 70%, at least 80%, at least 90%, or at least 100%; optionally, wherein the portion of the unmodified refined protein component that is deamidated is between about 30% and 100%, between about 50% and 100%, or between about 70% and 100%.

F9. The method of any one of clauses F1 to F8, wherein after at least partial mixing with an aqueous solution, the non-dairy analog: (a) exhibits no visible feathering for at least 5 minutes, 10 minutes, or 15 minutes after mixing; (b) exhibits no visible precipitation for at least 5 minutes, 10 minutes, or 15 minutes after mixing; (c) exhibits insubstantial precipitation for at least 5 minutes, 10 minutes, or 15 minutes after mixing; (d) exhibits less than 5%, 3%, 1% or 0.5% by weight precipitation; optionally, wherein the by weight precipitation is exhibited for at least 5 minutes, 10 minutes, or 15 minutes after mixing; (e) exhibits less than 5%, 3%, 1% or 0.5% by volume precipitation; optionally, wherein the by volume precipitation is exhibited for at least 5 minutes, 10 minutes, or 15 minutes after mixing; and/or (f) is solubilized and/or dispersed, or substantially solubilized and/or substantially dispersed, for at least 5 minutes, 10 minutes, or 15 minutes after mixing.

While the foregoing disclosure of the present invention has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the spirit and purview of this disclosure and the appended claims. Further, one of skill in the art will recognize a number of equivalent methods and procedure to those described herein. All such equivalents are to be understood to be within the scope of the present disclosure and are covered by the appended claims.

Additional embodiments of the invention are set forth in the following claims.

The disclosures of all publications, patent applications, patents, or other documents mentioned herein are expressly incorporated by reference in their entirety for all purposes to the same extent as if each such individual publication, patent, patent application or other document were individually specifically indicated to be incorporated by reference herein in its entirety for all purposes and were set forth in its entirety herein. In case of conflict, the present specification, including specified terms, will control.

1. A non-dairy analog, comprising:
(a) a refined protein component, at least a portion of which is a deamidated refined protein component;
(b) at least one lipid from a non-animal natural source;
(c) at least one emulsifier;
(d) water; and
(e) having a pH of between 4.0 and 10.

2. The non-dairy analog of claim 1, wherein:
(a) the refined protein component is at least 0.5% by weight of the non-dairy analog, and at least 10% by weight of the refined protein component is a deamidated refined protein component;
(b) the lipid is between 1% by weight and 10% by weight of the non-dairy analog; or
(c) the emulsifier is between 0.01% by weight and 5% by weight of the non-dairy analog.

3-6. (canceled)

7. The non-dairy analog of claim 1, wherein the refined protein component is sourced from a plant, including a legume.

8. The non-dairy analog of claim 7, wherein the refined protein component is sourced from a pea plant or a pea protein.

9. (canceled)

10. The non-dairy analog of claim 1, wherein the non-dairy analog, after at least partial mixing with an acidic beverage exhibits no visible feathering for at least 5 minutes after mixing.

11-14. (canceled)

15. A method for producing a non-dairy analog, the method comprising:
(a) blending with water to generate a mixture:
(i) at least one lipid from a non-animal natural source, and
(ii) at least one refined protein component from a non-animal natural source, wherein at least a portion of the refined protein component comprises a deamidated refined protein; and
(b) emulsifying at least a portion of the mixture to provide a non-dairy analog.

16. The method of claim 15, wherein the deamidated refined protein component is deamidated by glutaminase treatment.

17. The method of claim 16, wherein the glutaminase treatment comprises incubation with at least 0.1 wt % glutaminase relative to the refined protein component.

18. The method of claim 15, wherein the refined protein component is sourced from a plant, including a legume.

19. The method of claim 15, wherein the refined protein component is sourced from a pea plant or a pea protein.

20. The method of claim 15, wherein,
(a) the refined protein component is at least 0.5% by weight of the non-dairy analog and at least 10% by weight of the refined protein component is a deamidated refined protein component;
(b) the lipid is between 1% by weight and 10% by weight of the non-dairy analog; or
(c) the emulsifier is between 0.01% by weight and 5% by weight of the non-dairy analog.

21-22. (canceled)

23. A beverage formulation comprising:
(a) a refined protein component, at least a portion of which is a deamidated refined protein component;
(b) water or carbonated water; and
(c) having a pH of between 6 and 9.

24. The beverage formulation of claim 23, wherein the deamidated refined protein component is at least 10% by weight of the total weight of the refined protein component.

25. The beverage formulation of claim 23, wherein the refined protein component comprises at least 0.2% by weight of the beverage formulation.

26. The beverage formulation of claim 23, wherein the beverage:
(a) exhibits less than 0.5% by weight precipitation of the refined protein component; or
(b) exhibits less than 0.5% by volume precipitation of the refined protein component.

27. The beverage formulation of claim 23, wherein the refined protein component is sourced from a plant, including a legume.

28. The beverage formulation of claim 23, wherein the refined protein component is sourced from a pea plant or a pea protein.

29. The beverage formulation of claim 23, wherein the beverage formulation further comprises:
(a) sugar or a carbohydrate;
(b) at least one vitamin or mineral;
(c) at least one lipid from a non-animal natural source; or
(d) at least one emulsifier, or a hydrocolloid or gum.

30. The beverage formulation of any one of claim 23, wherein the beverage formulation is selected from: a protein drink, a vitamin drink, a fruit juice drink, or a coffee or tea drink.

31. A method for preparing a non-dairy analog, the method comprising:
(a) generating an aqueous mixture at pH 7.5-8.5 and 35-60° C. comprising: an 0.5-5.0 wt % unmodified refined protein component from a non-animal natural source, and 0.3-1.0 wt % buffering salts;
(b) adding to the mixture 0.1-1.0 wt % of a glutaminase and mixing at 35-60° C. for at least 1 hour, whereby at least a portion of the unmodified refined protein component is deamidated;
(c) after mixing of step (b) adding to the mixture 2-10 wt % oil, 0.01-0.05 wt % gums, 0-1 wt % emulsifier, 0-1 wt % flavors and 0-2 wt % sugar; and
(d) heating the mixture to 90° C. and subjecting to homogenization.

32-39. (canceled)

40. The non-dairy analog of claim 1, wherein the refined protein component is at an acidic pH.

41. The non-dairy analog of claim 1, comprising milk, yogurt, pudding, ice cream, coffee creamer, heavy cream, whipping cream, sour cream, soft cheese or hard cheese.

42. The method of claim 15, wherein the refined protein component is at an acidic pH.
